(19) United States
(12) Patent Application Publication
(10) Pub. No.: US 2018/0100136 A1
(43) Pub. Date: Apr. 12, 2018

(54) Mutant Yarrowia Strain Capable of Degrading Galactose

(71) Applicant: Institut National de la Recherche Agronomique, Paris (FR)

(72) Inventors: Jean-Marc Nicaud, Trappes (FR); Zbigniew Lazar, Wroclaw (PL); Anne-Marie Crutz-Le Coq, Velizy (FR); Heber Gamboa-Melen-Dez, Paris (FR); Cécile Neuveglise, Auffargis (FR)

(73) Assignee: Institut National de la Recherche Agronomique, Paris (FR)

(21) Appl. No.: 15/578,504

(22) PCT Filed: Jun. 17, 2016
(86) PCT No.: PCT/EP2016/064063

§ 371 Date: Nov. 30, 2017

(30) Foreign Application Priority Data
Jun. 17, 2015 (EP) 15172495.2

(51) Int. Cl. C12N 1/16; C12P 7/64; C12P 7/48; C12N 9/12; C12N 9/90

(52) U.S. Cl. CPC C12N 1/16; C12P 7/64; C12P 7/48; C12N 9/1205; C12Y 501/03003; C12N 9/1241; C12Y 207/07012; C12N 9/90; C12Y 501/03002; C12Y 207/01005

(57) ABSTRACT

The invention relates to a method for obtaining a mutant Yarrowia yeast strain capable of growing on D-galactose as sole carbon source, comprising over expressing in said strain a galactokinase, a galactose-1-phosphate uridyl transferase, an UDP-glucose-4 epimerase and a galactose mutarotase. The invention also relates to a mutant Yarrowia strain obtained by said method.

The present invention relates to mutant Yarrowia strains capable of growing on galactose as carbon source and means for obtaining such mutant strains.

Compounds such as citric acid, lipids, or sugar alcohols are of industrial interest. As a consequence, using microorganisms such as Yarrowia lipolytica to produce them has spurred research around the world (Beopoulos et al., 2012; Tai and Stephanopoulos, 2013; Liu et al., 2014; Mirończuk et al., 2014). From an economic and social point of view, it is very important to identify raw materials that could serve as cheap and renewable substrates and that are not already in demand for food production. Therefore, at present, research efforts are focused on using plant biomass, which contains a diversity of sugars, for the production of these valuable compounds (Blazeck et al., 2014; Wang et al., 2014). One such sugar is D-galactose, a monosaccharide that is a C4 epimer of glucose. The polysaccharides found in plant cell walls (e.g., galactomannans), gums, hemicelluloses, and pectins are rich sources of galactose (Schädel et al., 2010; Christensen et al., 2011). Galactose also occurs naturally in milk: lactose is made up of galactose and glucose. Both milk and plant biomass are readily exploited by many microorganisms, including bacteria, yeasts, and fungi. These diverse species utilize only a few pathways to break down galactose. For example, the bacterium Azotobacter vinelandii uses the non-phosphorylative DeLey-Doudoroff pathway to metabolize galactose. Galactose is oxidized, forming galactonate, which is ultimately broken down into pyruvate and glyceraldehyde 3-phosphate (Wong and Yao, 1994). Another pathway, the oxido-reductive pathway, exists in filamentous fungi such as Aspergillus niger or Hypocrea jecorina. It involves a series of redox reactions that convert galactose to D-fructose (Gruben et al., 2012; Koivistoinen et al., 2012; Mojzita et al., 2012). However, the best-known and best-studied pathway is the Leloir pathway, which occurs in many organisms, including yeasts such as Saccharomyces cerevisiae and Kluyveromyces lactis (Meyer et al., 1991; Frey, 1996; Holden et al., 2003; Sellick et al., 2008). Via this phosphorylative pathway, D-galactose is converted to glucose-6-phosphate. In S. cerevisiae, β-D-galactose is first converted to its α-anomer by galactose mutarotase (scGAL10, YBR019C); only this anomeric form can be utilized by cells. Subsequently, α-D-galactose is phosphorylated by galactokinase (scGAL1, YBR020W), releasing galactose-1-phosphate. Then, galactose-1-phosphate uridyltransferase (scGAL7, YBR018C) converts this intermediate compound into UDP-galactose, simultaneously releasing glucose-1-phosphate. In the last step of the pathway, UDP-galactose is epimerized into UDP-glucose by UDP-galactose 4-epimerase (scGAL10, YBR019C). It is worth noting that, in S. cerevisiae and other ascomycetes, the epimerase and mutarotase domains are fused together but act independently (Slot and Rokas, 2010). Additionally, the mutarotase domain is not essential to galactose metabolism because the sugar anomers interconvert spontaneously in water (Bouffard et al., 1994). Phosphoglucomutase converts the glucose-1-phosphate released by the pathway to glucose-6-phosphate, an intermediate compound in glycolysis. However, because phosphorrglucomutase is also involved in glycogenolysis, it is not formally considered to be part of the Leloir pathway. In addition, the form of Leloir pathway regulation that exists in S. cerevisiae may be restricted to yeasts and not conserved in other ascomycetes (Hartl et al., 2012).

Y. lipolytica is a dimorphic ascomycete yeast that belongs to the subphylum Saccharomycotina (van der Walt and von Arx, 1980). Y. lipolytica is able to use a few monosaccharides as carbon sources, namely glucose, fructose, and mannose (Coelho et al., 2010; Michely et al., 2013). All the genes that encode putative structural Leloir pathway proteins are present in its genome, but the wild type strains of this species appear to be unable to use galactose as its sole carbon source (Slot and Rokas, 2010). The genes are not clustered as they are in S. cerevisiae and other ascomycetes and, furthermore, are located on different chromosomes (Slot and Rokas, 2010).

The inventors have found that Y. lipolytica carry and express all the Leloir pathway genes, which encode fully functional proteins that are involved in galactose utilization. When Y. lipolytica was grown on mixed media, containing both galactose and varying concentrations of glucose, it was able to metabolize galactose, including when glucose concentrations were higher than 4 g·L⁻¹. However, in such mixed media, glucose was still the preferred carbon source.

Surprisingly, a Y. lipolytica strain in which the four ylGAL genes were overexpressed, namely:
YlGAL1 (YALI0C13090g) coding for a galactokinase,
YlGAL7 (YALI0F23947g) coding for a galactose-1-phosphate uridyl transferase,
YlGAL10e (YALI0E26829g) coding for a UDP-glucose-4 epimerase, and
YlGAL10m (YALI0C09570g) coding for a galactose mutarotase,
   was able to efficiently use galactose as its sole carbon source. This quadruple mutant was used to successfully produce citric acid and lipids from galactose; the yield of these compounds was comparable or greater than that obtained for the parental strain (W29) grown on glucose media.

These results are surprising because efforts at engineering Y. lipolytica able to degrading sugars which are not reported as being naturally consumed by Y. lipolytica, such as xylose, by overexpressing endogenous putative genes involved in the sugar (e.g., xylose) degrading pathway, were not successful (see International Application WO 2013/192520).

The effective activation of the galactose utilization pathway in Y. lipolytica can therefore serve as an excellent starting point for further improving citric acid and lipid production from renewable substrates.

Accordingly, the present invention provides a method for obtaining a Yarrowia strain, preferably a Y. lipolytica strain, capable of growing on D-galactose as sole carbon source, wherein said method comprises overexpressing in said strain

a galactokinase (E.C 2.7.1.6) having at least 75% identity, or by order of increasing preference at least 78%, 80%, 85%, 90%, 92%, 95%, 96%, 97%, 98% or 99% identity, with the polypeptide of sequence SEQ ID NO: 2 (YALI_GAL1),

a galactose-1-phosphate uridyl transferase (E.C 2.7.7.12) having at least 65% identity, or by order of increasing preference at least 70%, 75%, 80%, 85%, 90%, 92%, 95%, 96%, 97%, 98% or 99% identity, with the polypeptide of sequence SEQ ID NO: 4 (YALI_GAL7),

an UDP-glucose-4 epimerase (E.C 5.1.3.2) having at least 85% identity, or by order of increasing preference at least 88%, 90%, 92%, 95%, 96%, 97%, 98% or 99% identity, with the polypeptide of sequence SEQ ID NO: 6 (YALI_GAL10E) and

a galactose mutarotase (E.C 5.1.3.3) having at least 45% identity, or by order of increasing preference at least 48%, 50%, 55%, 60%, 65%, 70%, 75%, 80%, 85%, 90%, 92%, 95%, 96%, 97%, 98% or 99% identity, with the polypeptide of sequence SEQ ID NO: 8 (YALI_GAL10M).

Unless otherwise specified, the percent of identity between two protein sequences which are mentioned herein is calculated from the best alignment of the two sequences using the BLAST program (Tatusova and Madden, 1999) with the default parameters (open gap penalty=2; extension gap penalty=5; matrix=BLOSUM 62).

The term overexpressing an enzyme in a Yarrowia strain, herein refers to artificially increasing the quantity of said enzyme produced in a Yarrowia strain compared to a reference (control) Yarrowia strain wherein said enzyme is noₜ overexpressed. This term also encompasses expression of an enzyme in a Yarrowia strain which does not naturally contain a gene encoding said enzyme.

An advantageous method for overexpressing an enzyme in a Yarrowia strain comprises introducing into the genome of said Yarrowia strain a DNA construct comprising a nucleotide sequence encoding said enzyme, placed under the control of a promoter.

Nucleotide sequences encoding YALI_GAL1, YALI_GAL7, YALI_GAL10E and YALI_GAL10M are provided in SEQ ID NO: 1, SEQ ID NO: 3, SEQ ID NO: 5 and SEQ ID NO: 7 respectively.

Methods for determining whether an enzyme has a galactokinase (E.C2.7.1.6) activity are known in the art. By way of example, galactokinase activity can be measured by quantifying formation of galactose1-phosphate from C14-galactose, as described in Schell and Wilson (1977).

Methods for determining whether an enzyme has a galactose-1-phosphate uridyl transferase (E.C 2.7.7.12) activity are known in the art. By way of example, galactose-1-phosphate uridyl transferase activity can be measured by quantifying the release of glucose-1-phosphate from UDP-Glucose, as described in Segawa and Fukasawa (1979).

Methods for determining whether an enzyme has an UDP-glucose-4 epimerase (E.C 5.1.3.2) activity are known in the art. By way of example, UDP-glucose-4 epimerase activity can be measured by quantifying the release of UDP-Glucuronic acid from UDP-glucose which was previously formed from UDP-galactose, as described in Majumdar et al. (2004).

Methods for determining whether an enzyme has a galactose mutarotase (E.C5.1.3.3) activity are known in the art. By way of example, galactose mutarotase activity can be measured polarimetrically by the formation of β-D-glucose or β-D-galactose from α-D-glucose or α-D-galactose, as described in Majumdar et al. (2004).

Advantageously, at least one, or by order of increasing preference at least 2, 3 or 4 of the enzymes as defined above (i.e., galactokinase, galactose-1-phosphate uridyl transferase, UDP-glucose-4 epimerase and galactose mutarotase), preferably the 4 enzymes as defined above, are from a Yarrowia strain, preferably selected from the group consisting of Y. lipolytica, Y. galli, Y. yakushimensis, Y. alimentaria and Y. phangngensis, provided that said strain naturally comprises the enzyme as defined above, more preferably from a Y. lipolytica strain.

At least one, or by order of increasing preference at least 2, 3 or 4 of the enzymes as defined are endogenous enzymes, i.e., are from the Yarrowia strain in which the overexpression is performed.

In a preferred embodiment, the galactokinase having at least 75% identity with the polypeptide of sequence SEQ ID NO: 2 (YALI_GAL1) has the consensus amino acid sequence SEQ ID NO: 9. This sequence SEQ ID NO: 9 corresponds to the consensus amino acid sequence obtained by aligning the galactokinase from the strains Y. lipolytica CLIB122 (YALI_GAL1 of SEQ ID NO: 2), Y. galli CBS 9722 (YAGA_GAL1 of SEQ ID NO: 10), Y. yakushimensis CBS 10253 (YAYA_GAL1 of SEQ ID NO: 11), Y. alimentaria CBS 10151 (YAAL_GAL1 of SEQ ID NO: 12) and Y. phangngensis CBS 10407 (YAPH_GAL1 of SEQ ID NO: 13).

Advantageously, the galactokinase having at least 75% identity with the polypeptide of sequence SEQ ID NO: 2 is selected from the group consisting of SEQ ID NO: 2, 10, 11, 12 and 13, preferably SEQ ID NO: 2.

The galactokinase enzymes of SEQ ID NO: 2 (YALI_GAL1), SEQ ID NO: 10 (YAGA_GAL1), SEQ ID NO: 11 (YAYA_GAL1), SEQ ID NO: 12 (YAAL_GAL1) and SEQ ID NO: 13 (YAPH_GAL1) have respectively 100%, 94.33%, 92.89%, 78.15% and 78.97% identity with the polypeptide of sequence SEQ ID NO: 2 (YALI_GAL1).

In another preferred embodiment, the galactose-1-phosphate uridyl transferase having at least 65% identity with the polypeptide of sequence SEQ ID NO: 4 has the consensus amino acid sequence SEQ ID NO: 14. This sequence SEQ ID NO: 14 corresponds to the consensus amino acid sequence obtained by aligning the galactose-1-phosphate uridyl transferase from the strains Y. lipolytica CLIB122 (YALI_GAL7 of SEQ ID NO: 4), Y. galli CBS 9722 (YAGA_GAL7 of SEQ ID NO: 15), Y. yakushimensis CBS 10253 (YAYA_GAL7 of SEQ ID NO: 16), Y. alimentaria CBS 10151 (YAAL_GAL7 of SEQ ID NO: 17) and Y. phangngensis CBS 10407 (YAPH_GAL7 of SEQ ID NO: 18).

Advantageously, the galactose-1-phosphate uridyl transferase having at least 65% identity with the polypeptide of sequence SEQ ID NO: 4 is selected from the group consisting of SEQ ID NO: 4, 15, 16, 17 and 18, preferably SEQ ID NO: 4.

The galactose-1-phosphate uridyl transferase enzymes of SEQ ID NO: 4 (YALI_GAL7), SEQ ID NO: 15 (YAGA_GAL7), SEQ ID NO: 16 (YAYA_GAL7), SEQ ID NO: 17 (YAAL_GAL7) and SEQ ID NO: 18 (YAPH_GAL7) have respectively 100%, 91.17%, 87.36%, 70.65% and 70.83% identity with the polypeptide of sequence SEQ ID NO: 4 (YALI_GAL7).

In another preferred embodiment, the UDP-glucose-4 epimerase having at least 85% identity with the polypeptide of sequence SEQ ID NO: 6 (YALI_GAL10E) has the consensus amino acid sequence SEQ ID NO: 19. This sequence SEQ ID NO: 19 corresponds to the consensus amino acid sequence obtained by aligning the UDP-glucose-4 epimerase from the strains Y. lipolytica CLIB122 (YALI_GAL10E of SEQ ID NO: 6), Y. galli CBS 9722 (YAGA_GAL10E of SEQ ID NO: 20), Y. yakushimensis CBS 10253 (YAYA_GAL10E of SEQ ID NO: 21), Y. alimentaria CBS 10151 (YAAL_GAL10E of SEQ ID NO: 22) and Y. phangngensis CBS 10407 (YAPH_GAL10E of SEQ ID NO: 23).

Advantageously, the UDP-glucose-4 epimerase having at least 85% identity with the polypeptide of sequence SEQ ID NO: 6 is selected from the group consisting of SEQ ID NO: 6, 20, 21, 22 and 23, preferably SEQ ID NO: 6.

The UDP-glucose-4 epimerase enzymes of SEQ ID NO: 6 (YALI_GAL10E), SEQ ID NO: 21 (YAGA_GAL10E), SEQ ID NO: 22 (YAYA_GAL10E), SEQ ID NO: 23 (YAAL_GAL10E) and SEQ ID NO: 24 (YAPH_GAL10E) have respectively 100%, 97.56%, 93.22%, 90.96% and 88.59% identity with the polypeptide of sequence SEQ ID NO: 6 (YALI_GAL10E).

In another preferred embodiment, the amino acid sequence of the galactose mutarotase having at least 45% identity with the polypeptide of sequence SEQ ID NO: 8 (YALI_GAL10M) comprises, from the N-terminus to the C-terminus, the polypeptide fragments of SEQ ID NO: 24, 25 and 26. These fragments of SEQ ID NO: 24, 26 and 26 have been obtained by aligning the galactose mutarotase from the strains Y. lipolytica CLIB122 (YALI_GAL10M of SEQ ID NO: 8), Y. galli CBS 9722 (YAGA_GAL10M of SEQ ID NO: 27), Y. yakushimensis CBS 10253 (YAYA_GAL10M of SEQ ID NO: 28) and Y. alimentaria CBS 10151 (YAAL_GAL10M of SEQ ID NO: 29).

Advantageously, the galactose mutarotase having at least 45% identity with the polypeptide of sequence SEQ ID NO: 8 (YALI_GAL10M) is selected from the group consisting of SEQ ID NO: 8, 27, 28 and 29, preferably SEQ ID NO: 8.

The galactose mutarotase enzymes of SEQ ID NO: 8 (YALI_GAL10M), SEQ ID NO: 27 (YAGA_GAL10M), SEQ ID NO: 28 (YAYA_GAL10M) and SEQ ID NO: 29 (YAAL_GAL10M) have respectively 100%, 73.43%, 67.38% and 48.76% identity with the polypeptide of sequence SEQ ID NO: 8 (YALI_GAL10M),

Advantageously, said Yarrowia strain is auxotrophic for leucine (Leu) and optionally for the decarboxylase orotidine-5′-phosphate (Ura-).

Said Yarrowia strain can also be a mutant Yarrowia strain wherein the expression or activity of the endogenous isoforms of acyl-coenzymeA oxidases (AOX, EC 1.3.3.6) involved, at least partially, in the β-oxidation of fatty acids, is inhibited. In Yarrowia, 6 genes (POX1, POX2, POX3, POX4, POX5 and POX6) encode these isoforms. Said inhibition of the expression or activity can be total or partial. Total or partial inhibition of the expression or activity of these enzymes leads to accumulation by yeast of dodecanedioic acid without use of accumulated fat. More particularly, the coding sequence of the genes POX1-6 and the peptide sequence of AOX1-6 from Y. lipolytica CLIB122 are available in either at Génolevures database (http://genolevures.org/) or at GRYC database (http://gryc.inra.fr/) or in GenBank database under the following accession numbers or names: POX1/AOX1=YALI0E32835g/YALI0E32835p, POX2/AOX2=YALI0F10857g/YALI0F10857p; POX3/AOX3=YALI0D24750g/YALI0D24750p; POX4/AOX4=YALI0E27654g/YALI0E27654p; POX5/AOX5=YALI0C23859g/YALI0C23859p; POX6/AOX6=YALI0E06567g/YALI0E06567p. The peptide sequences of the acyl-CoA oxidases of Y. lipolytica have 45% identity or 50% similarity with those from other yeasts. The degree of identity between the acyl-CoA oxidases varies from 55% to 70% (or from 65 to 76% similarity) (see International Application WO 2006/064131). A method of inhibiting the expression of the 6 endogenous AOX in a Y. lipolytica strain is described in Beopoulos et al., 2008 and International Applications WO 2006/064131, WO 2010/004141 and WO 2012/001144.

The Yarrowia strain can further comprise other mutations such as those described in International Applications WO 2006/064131, WO 2010/004141, WO 2012/001144, WO 2014/178014 and WO 2014/136028 which are useful for obtaining a fatty acids or citric acid producing yeast strain.

In particular, the Yarrowia strain can be genetically modified to improve lipid accumulation. Said Yarrowia strain having improved properties for lipid accumulation can be a mutant Yarrowia strain, preferably a Y. lipolytica mutant strain, wherein at least one protein, preferably at least one endogenous protein, selected from the group consisting of an acyl-CoA:diacylglycerol acyltransferase 2 (encoded by DGA1), an acyl-CoA:diacylglycerol acyltransferase 1 (encoded by DGA2), a glycerol-3-phosphate dehydrogenase NAD+ (encoded by GPD1), an acetyl-CoA carboxylase (encoded by ACC1) and a hexokinase (encoded by HXK1; EC number: 2.7.1.1; YALI0B22308g in Y. lipolytica) is overexpressed, and/or the expression or activity of at least one endogenous protein selected from the group consisting of the glycerol 3-phosphate dehydrogenase (encoded by GUT2), the triglyceride lipase (encoded by TGL4) and the peroxin 10 (encoded by PEX10) is inhibited.

Advantageously, the protein hexokinase (encoded by HXK1; EC number: 2.7.1.1; YALI0B22308g in Y. lipolytica) is overexpressed in said mutant yeast strain, preferably in said mutant Y. lipolytica strain.

Advantageously, the 5 proteins, acyl-CoA:diacylglycerol acyltransferase 2, acyl-CoA:diacylglycerol acyltransferase 1, glycerol-3-phosphate dehydrogenase NAD+, acetyl-CoA carboxylase and hexokinase, are overexpressed, and the expression or activity of the 3 endogenous proteins, glycerol 3-phosphate dehydrogenase, triglyceride lipase and peroxin, are inhibited in said mutant yeast strain.

A method of overexpressing the endogenous genes DGA1, DGA2, GPD1 and ACC1 and inhibiting the expression or activity of the endogenous genes GUT2, TGL4 and PEX10 in a Y. lipolytica strain is described in International Application WO 2014/136028.

A method of overexpressing the endogenous genes DGA2, GPD1 and HXK1, and inhibiting the expression or activity of the endogenous gene TGL4 in a Y. lipolytica strain is described in Lazar et al., 2014.

Overexpression of an enzyme as defined above—which can be an endogenous, ortholog or heterologous enzyme—in a Yarrowia strain according to the present invention can be obtained in various ways by methods known per se.

Overexpression of an enzyme as defined in the present invention may be performed by placing one or more (preferably two or three) copies of the coding sequence (CDS) of the sequence encoding said enzyme under the control of appropriate regulatory sequences. Said regulatory sequences include promoter sequences, located upstream (at 5′ position) of the ORF of the sequence encoding said enzyme, and terminator sequences, located downstream (at 3′ position) of the ORF of the sequence encoding said enzyme.

Promoter sequences that can be used in yeast are well known to those skilled in the art and may correspond in particular to inducible or constitutive promoters. Examples of promoters which can be used according to the present invention, include the promoter of a Y. lipolytica gene which is strongly repressed by glucose and is inducible by the fatty acids or triglycerides such as the promoter of the POX2 gene encoding the acyl-CoA oxidase 2 (AOX2) of Y. lipolytica and the promoter of the LIP2 gene described in International Application WO 01/83773. One can also use the promoter of the FBA1 gene encoding the fructose-bisphosphate aldolase (see Application US 2005/0130280), the promoter of the GPM gene encoding the phosphoglycerate mutase (see International Application WO 2006/0019297), the promoter of the YAT1 gene encoding the transporter ammonium (see Application US 2006/0094102), the promoter of the GPAT gene encoding the O-acyltransferase glycerol-3-phosphate (see Application US 2006/0057690), the promoter of the TEF gene (Müller et al., 1998; Application US 2001/6265185), the hybrid promoter hp4d (described in International Application WO 96/41889), the hybrid promoter XPR2 described in Mazdak et al. (2000) or the hybrid promoters UAS1-TEF or UAStef-TEF described in Blazeck et al. (2011, 2013, 2014).

Advantageously, the promoter is the promoter of the TEF gene.

Terminator sequences that can be used in yeast are also well known to those skilled in the art. Example of terminator sequences which can be used according to the present invention include the terminator sequence of the PGK1 gene and the terminator sequence of the LIP2 gene described in International Application WO 01/83773.

The nucleotide sequence of the coding sequences of the heterologous genes can be optimized for expression in yeast by methods well known in the art (see for review Hedfalk, 2012).

Overexpression of an endogenous enzyme as defined above can be obtained by replacing the sequences controlling the expression of said endogenous enzyme by regulatory sequences allowing a stronger expression, such as those described above. The skilled person can replace the copy of the gene encoding an endogenous enzyme in the genome, as well as its own regulatory sequences, by genetically transforming the yeast strain with a linear polynucleotide comprising the ORF of the sequence coding for said endogenous enzyme under the control of regulatory sequences such as those described above. Advantageously, said polynucleotide is flanked by sequences which are homologous to sequences located on each side of said chromosomal gene encoding said endogenous enzyme. Selection markers can be inserted between the sequences ensuring recombination to allow, after transformation, to isolate the cells in which integration of the fragment occurred by identifying the corresponding markers. Advantageously also, the promoter and terminator sequences belong to a gene different from the gene encoding the endogenous enzyme to be overexpressed in order to minimize the risk of unwanted recombination into the genome of the yeast strain.

Overexpression of an endogenous enzyme as defined above can also be obtained by introducing into the Yarrowia strain extra copies of the gene encoding said endogenous enzyme under the control of regulatory sequences such as those described above. Said additional copies encoding said endogenous enzyme may be carried by an episomal vector, that is to say capable of replicating in Yarrowia. Preferably, these additional copies are carried by an integrative vector, that is to say, integrating into a given location in the Yarrowia genome (Madzak et al., 2004). In this case, the polynucleotide comprising the gene encoding said endogenous enzyme under the control of regulatory regions is integrated by targeted integration. Said additional copies can also be carried by PCR fragments whose ends are homologous to a given locus of Yarrowia, allowing integrating said copies into the Yarrowia genome by homologous recombination. Said additional copies can also be carried by auto-cloning vectors or PCR fragments, wherein the ends have a zeta region absent from the genome of the yeast, allowing the integration of said copies into Yarrowia genome by random insertion as described in Application US 2012/0034652.

Targeted integration of a gene into the genome of a Yarrowia cell is a molecular biology technique well known to those skilled in the art: a DNA fragment is cloned into an integrating vector, introduced into the cell to be transformed, wherein said DNA fragment integrates by homologous recombination in a targeted region of the recipient genome (Orr-Weaver et al., 1981).

Methods for transforming yeast are also well known to those skilled in the art and are described, inter alia, by Ito et al. (1983), Klebe et al., (1983) and Gysler et al., (1990).

Any gene transfer method known in the art can be used to introduce a gene encoding an enzyme. Preferably, one can use the method with lithium acetate and polyethylene glycol described by Gaillardin et al., (1987) and Le Dall et al., (1994).

The present invention also provides means for carrying out said overexpression.

This includes, in particular, recombinant DNA constructs for expressing the four enzymes as defined above in a Yarrowia cell. These DNA constructs can be obtained and introduced in said Yarrowia strain by the well-known techniques of recombinant DNA and genetic engineering.

Recombinant DNA constructs of the invention include in particular expression cassettes, comprising a polynucleotide encoding 1, 2, 3 or 4 of the enzymes as defined above, each polynucleotide encoding an enzyme being under the control of a promoter functional in a Yarrowia cell as defined above.

The expression cassettes generally also include a transcriptional terminator, such as those describes above. They may also include other regulatory sequences, such as transcription enhancer sequences.

Recombinant DNA constructs of the invention also include recombinant vectors containing expression cassettes comprising a polynucleotide encoding 1, 2, 3 or 4 of the enzymes as defined above, each polynucleotide encoding an enzyme being under transcriptional control of a suitable promoter.

Recombinant vectors of the invention may also include other sequences of interest, such as, for instance, one or more marker genes, which allow for selection of transformed Yarrowia cells.

The invention also comprises host cells containing a recombinant DNA construct of the invention. These host cells can be prokaryotic cells (such as bacteria cells) or eukaryotic cells, preferably yeast cells.

The invention also provides a method for obtaining a mutant Yarrowia strain, preferably a mutant Y. lipolytica strain, capable of growing on D-galactose as carbon source as defined above, comprising transforming a Yarrowia cell with 1, 2, 3 or 4 recombinant DNA constructs as defined above for expressing the 4 enzymes as defined above.

As used herein the term “1, 2, 3 or 4 recombinant DNA constructs for expressing the 4 enzymes” refers to:

a single recombinant DNA construct for expressing the 4 enzymes as defined above (the coding sequences of the 4 enzymes are under the control of a suitable promoter respectively),

two recombinant DNA constructs for expressing the 4 enzymes as defined above, including
a recombinant DNA construct for expressing 1 enzyme and a recombinant DNA construct for expressing the 3 other enzymes (the coding sequences of the 3 enzymes are under the control of a suitable promoter respectively), or
a recombinant DNA construct for expressing 2 enzymes and a recombinant DNA construct for expressing the 2 other enzymes (the coding sequences of the 2 enzymes are under the control of a suitable promoter respectively),

three recombinant DNA constructs for expressing the 4 enzymes as defined above, including a recombinant DNA construct for expressing 1 enzyme, a recombinant DNA construct for expressing another enzyme and a recombinant DNA construct for expressing the 2 other enzymes (the coding sequences of the 2 enzymes are under the control of a suitable promoter respectively), or

four recombinant DNA constructs for expressing the 4 enzymes respectively.

The invention also comprises a Yarrowia strain, preferably a Y. lipolytica strain, genetically transformed with 1, 2, 3 or 4 recombinant DNA constructs for expressing the 4 enzymes as defined above, and overexpressing said 4 enzymes as defined above. In said mutant (transgenic) Yarrowia strain 1, 2, 3 or 4 recombinant DNA constructs for expressing the 4 enzymes as defined above is/are comprised in a transgene stably integrated in the Yarrowia genome, so that it is passed onto successive yeast generations. Thus the mutant (transgenic) Yarrowia strain of the invention includes not only the Yarrowia cell resulting from the initial transgenesis, but also their descendants, as far as they contain 1, 2, 3 or 4 recombinant DNA constructs for expressing the 4 enzymes as defined above. The overexpression of the 4 enzymes as defined above in said Yarrowia strains provides them an ability to grow on D-galactose as carbon source, when compared with a Yarrowia strain devoid of said transgene(s).

The present invention also comprises a mutant Yarrowia strain as defined above, preferably a mutant Y. lipolytica strain, wherein a galactokinase having at least 75% identity with the polypeptide of sequence SEQ ID NO: 2 (YALI_GAL1), a galactose-1-phosphate uridyl transferase having at least 65% identity with the polypeptide of sequence SEQ ID NO: 4 (YALI_GAL7), an UDP-glucose-4 epimerase having at least 85% identity with the polypeptide of sequence SEQ ID NO: 6 (YALI_GAL10E) and a galactose mutarotase having at least 45% identity with the polypeptide of sequence SEQ ID NO: 8 (YALI_GAL10M) as defined above. This mutant Yarrowia strain is obtainable by a method of the invention and contains 1, 2, 3 or 4 recombinant DNA constructs for expressing the 4 enzymes of the invention.

The present invention further comprises a mutant Yarrowia strain as defined above, preferably a mutant Y. lipolytica strain, comprising, stably integrated in its genome, 1, 2, 3 or 4 recombinant DNA constructs for expressing the 4 enzymes as defined above.

The present invention also provides the use of a mutant Yarrowia strain, preferably a mutant Y. lipolytica strain, as defined above for producing lipids or citric acid from a medium comprising D-galactose.

As used herein, the term producing lipids or citric acid refers to the accumulation and optionally secretion of lipids or citric acid.

The present invention also provides a method of producing lipids or citric acid, comprising a step of growing a mutant Yarrowia strain, preferably a mutant Y. lipolytica strain, of the invention on D-galactose.

Methods for extracting and purifying lipids or citric acid produced by cultured yeast strains are well known to those skilled in the art (Papanikolaou et al., 2001, 2002 and 2008; André et al., 2009). For example, the total lipids can be extracted according to the method described by Papanikolaou et al., 2001, and fractionated according to the methods described by Guo et al., 2000 and Fakas et at , 2006.

The present invention also provides an isolated enzyme selected from the group consisting of

a galactokinase (E.C 2.7.1.6.) of SEQ ID NO: 2, 10, 11, 12 or 13,

a galactose-1-phosphate uridyl transferase (E.C 2.7.7.12) of SEQ ID NO: 4, 15, 16 or 17,

an UDP-glucose-4 epimerase (E.C 5.1.3.2) of SEQ ID NO: 6, 20, 21, 22 or 23, and

a galactose mutarotase (E.C 5.1.3.3) of SEQ ID NO: 8, 27, 28 or 29.

The present invention will be understood more clearly from the further description which follows, which refers to non-limitative examples illustrating the overexpression of Gal11, Gal7, Gal10E and Gal10M in Y. lipoytica, as well as to the appended figures.

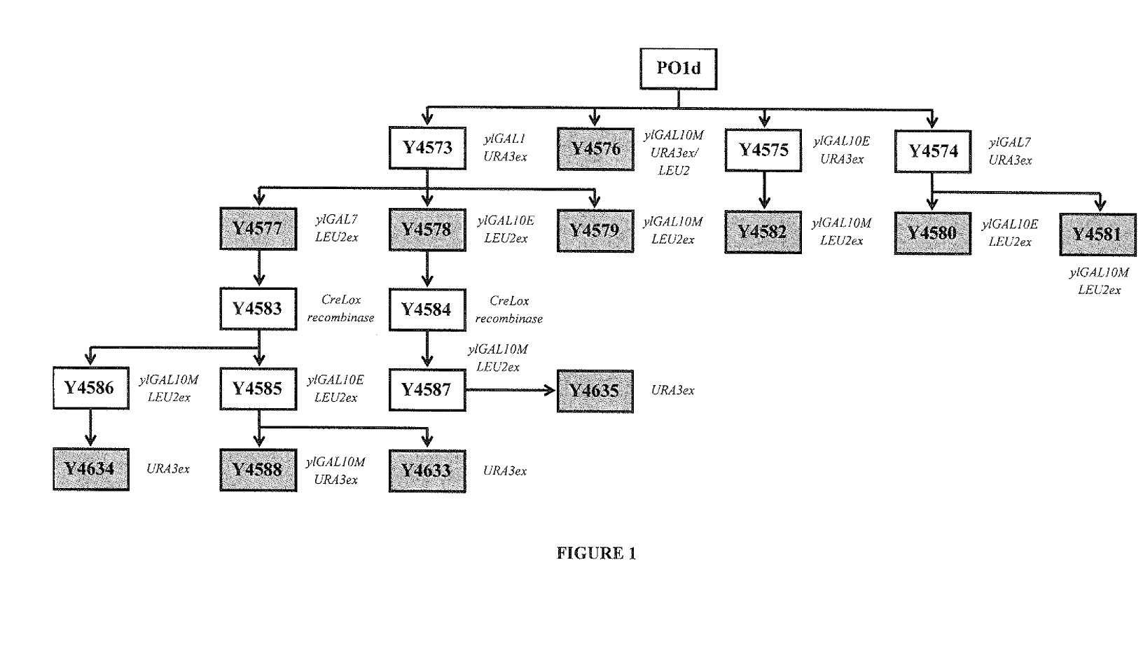

FIG. 1: Construction of Y. lipolytica strains overexpressing ylGAL genes. The auxotrophic PO1d strain was used as the acceptor strain. The genes were inserted one by one to create strains that overexpressed different combinations of the ylGAL genes; URA3ex and LEU2ex were used as selection markers. To recover prototrophy in some of the Y. lipolytica strains (gray squares), cassettes containing the URA3 excisable marker or the purified SalI fragment of a pINA62 plasmid that contained the LEU2 gene were inserted into the transformants' genomes (Gaillardin and Ribet, 1987). In turn, to recover URA3 and LEU2 auxotrophy, which was necessary to continue the transformation process, a JME547 plasmid containing Cre-Lox recombinase was used (Fickers et al., 2003).

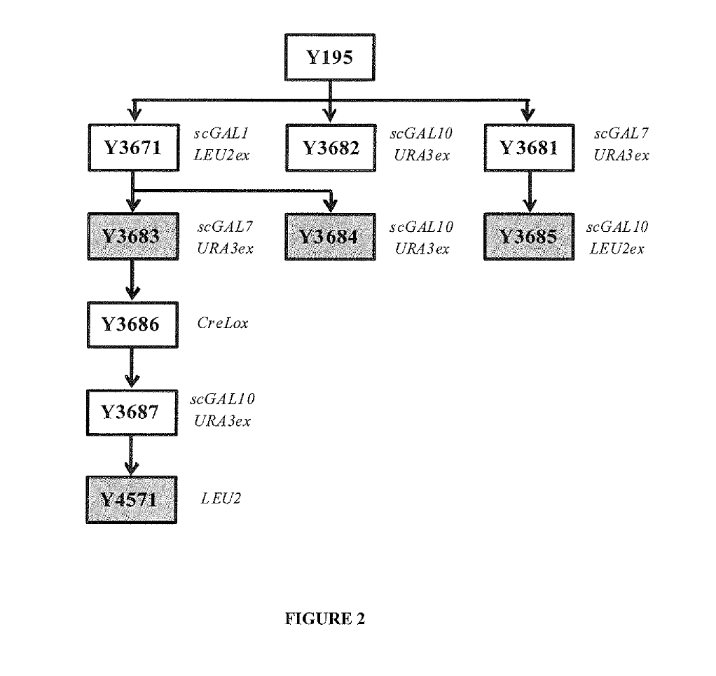

FIG. 2: Construction of the Y. lipolytica strains in which the scGAL genes were overexpressed. The auxotrophic PO1d strain was used as the acceptor strain. The genes were inserted one by one to create strains that overexpressed different combination of the scGAL genes; URA3ex and LEU2ex were used as selection markers. To recover auxotrophies necessary to continuing the transformation process, a JME547 plasmid containing Cre-Lox recombinase was used to transform Y. lipolytica Y3683, thus generating strain Y3686 (Fickers et al., 2003). In turn, to recover prototrophy in the Y. lipolytica Y3687 strain, a purified SalI fragment of the pINA62 plasmid that contained the LEU2 gene was introduced (Gaillardin and Ribet, 1987).

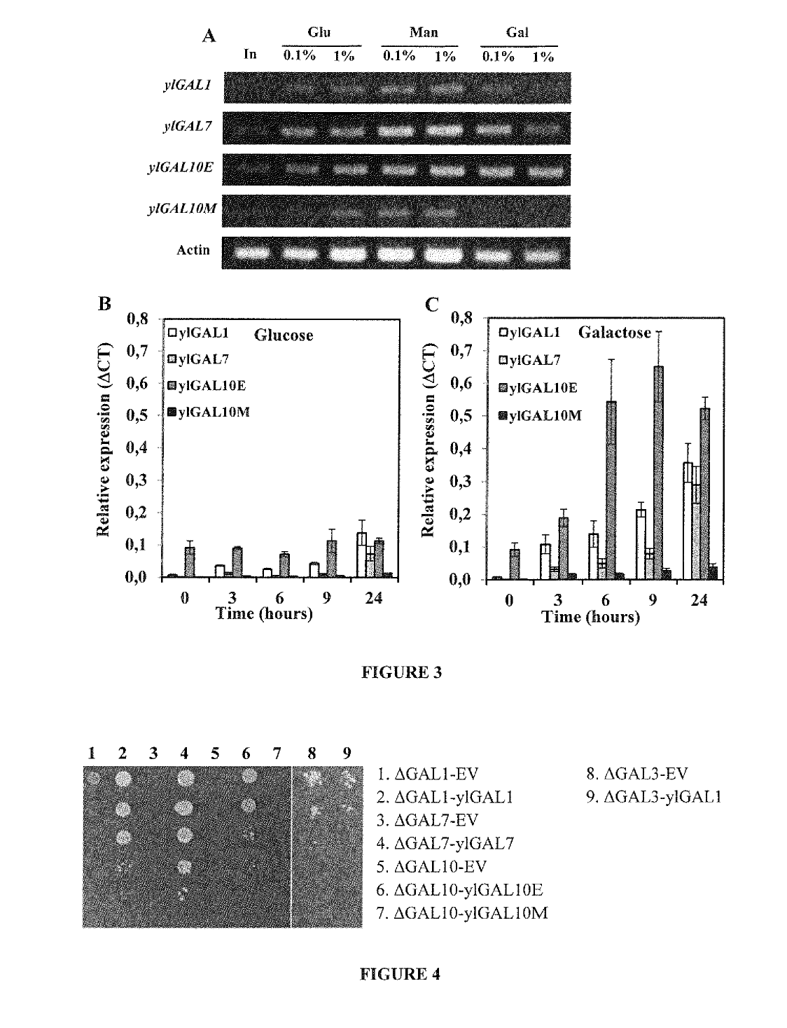

FIG. 3: Expression profiles of ylGAL genes in Y. lipolytica W29. Cells were incubated for 3 hours in YNB medium containing 0.1% or 1.0% glucose, mannose, or galactose (A). Kinetics of ylGAL gene expression in Y. lipolytica W29 grown in YNB medium containing 1% glucose (B) or 1% galactose (C). Gene expression levels were normalized based on the expression levels of the actin gene (ΔCT). Abbreviation: In—inoculum.

FIG. 4: Complementation of GAL gene deletion in S. cerevisiae using GAL homologs found in Y. lipolytica. EV—strains complemented for uracil deletion using a pRS426TEF empty vector.

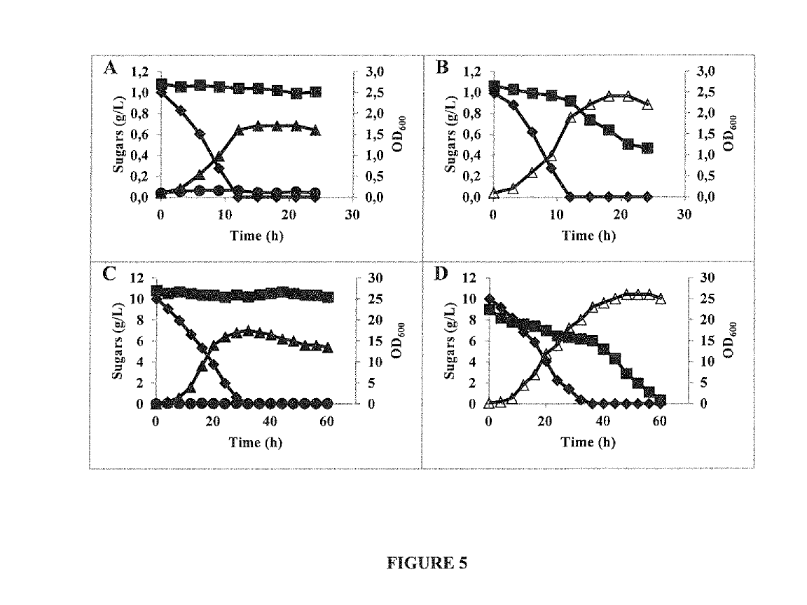

FIG. 5: Y. lipolytica W29 growth and sugar consumption over time in YNB medium containing glucose and galactose. (A) YNB medium containing only 0.1% glucose or 0.1% galactose; (B) YNB medium containing a mixture of 0.1% glucose and 0.1% galactose; (C) YNB medium containing only 1% glucose or 1% galactose; (D) YNB medium containing a mixture of 1% glucose and 1% galactose. Symbols: glucose (♦), galactose (▪), and optical density of cells growing in medium containing glucose (▴), galactose (●), or a mixture of glucose and galactose (Δ).

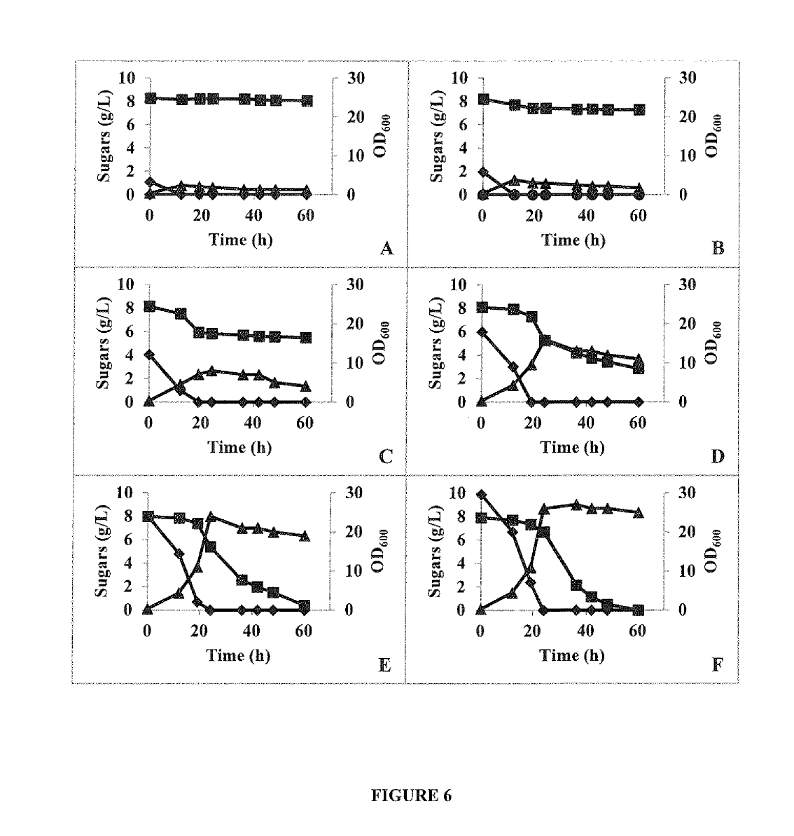

FIG. 6: Comparison of Y. lipolytica W29 growth and sugar consumption over time in YNB medium containing 1% galactose and different concentrations of glucose: 0.1% (A); 0.2% (B); 0.4% (C); 0.6% (D); 0.8% (E); 1.0% (F). Symbols: glucose (♦), galactose (▪), and OD₆₀₀ (▴).

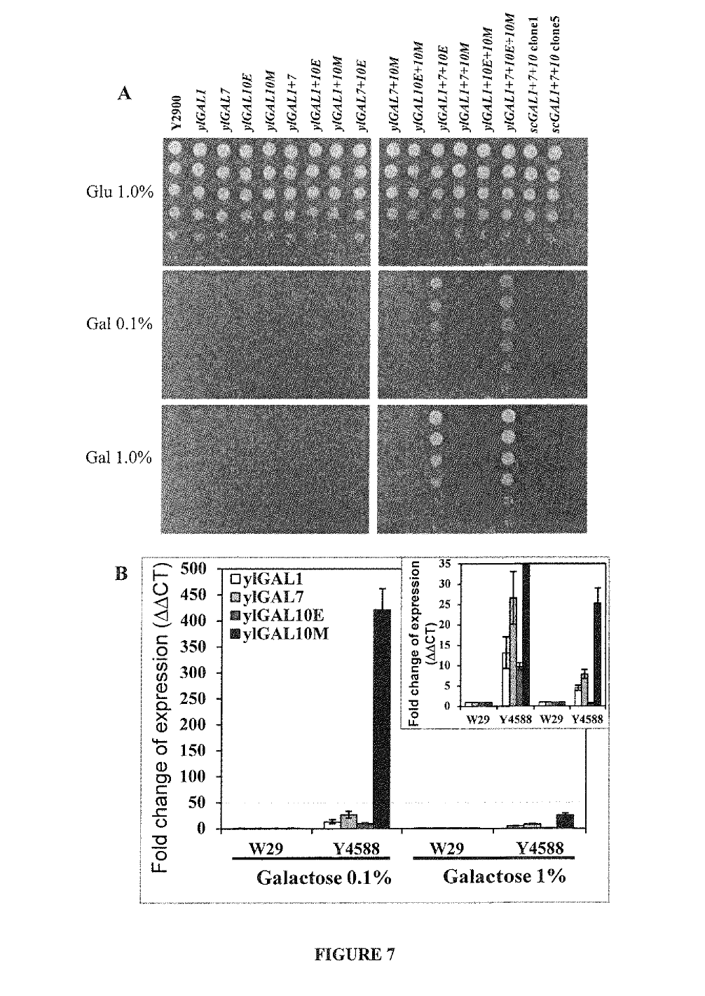

FIG. 7: Functional analysis of Leloir pathway overexpression in Y. lipolytica and fold change in the expression of the ylGAL genes. Growth of different Y. lipolytica transformants overexpressing ylGAL and scGAL genes on plates of YNB medium containing glucose or galactose (A). Fold change in the expression of ylGAL genes in the quadruple mutant, Y4588 (in which all the Leloir genes were overexpressed), relative to Y. lipolytica W29 (the wild-type strain) (B); yeast were incubated for 3 hours in YNB medium containing 0.1% or 1.0% galactose. Gene expression levels were normalized based on the expression of the actin gene.

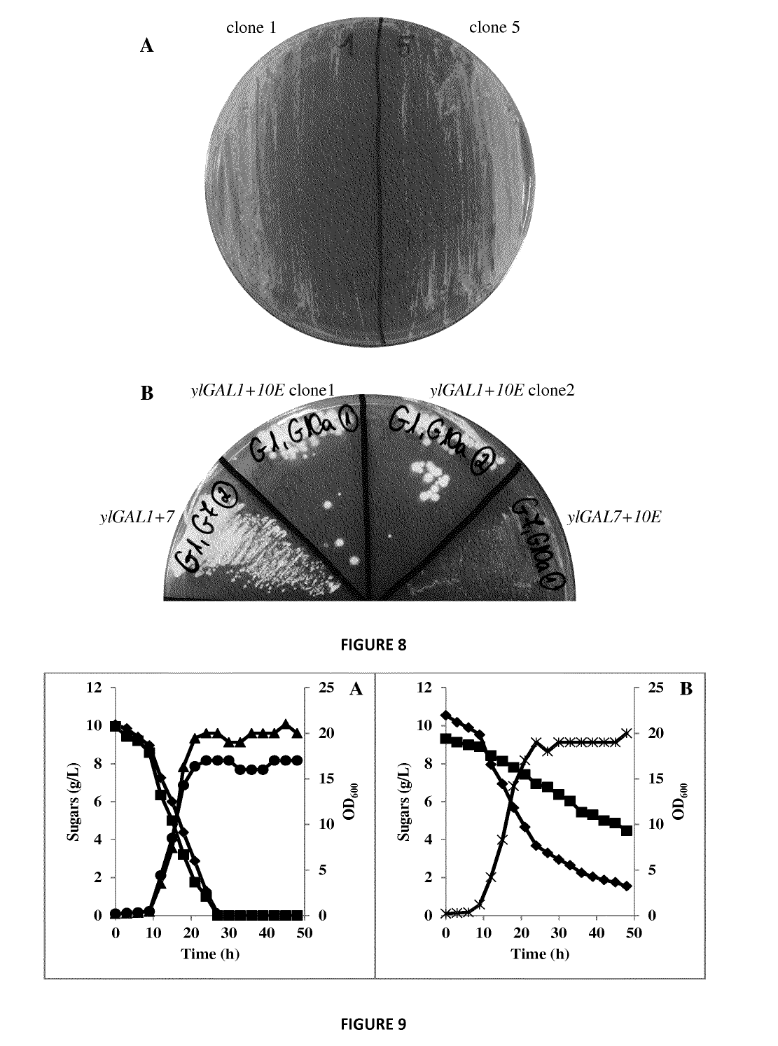

FIG. 8: Overexpression of scGAL and ylGAL genes in Y. lipolytica. Growth of Y. lipolytica transformants containing scGAL genes (scGAL1,7,10) on YNB medium containing 1% galactose (A); yeast were incubated for 1 week at 28° C. Growth of Y. lipolytica transformants overexpressing different combinations of ylGAL genes on YNB medium containing 1% galactose (B); yeast were incubated for 2 weeks at 28° C.

FIG. 9: Y. lipolytica Y4588 growth and sugar consumption over 48 h in YNB medium containing only 1% glucose, only 1% galactose (A), or a mixture of both 1% glucose and 1% galactose (B). Symbols: glucose (♦), galactose (▪), OD₆₀₀ in glucose (▴), OD₆₀₀ in galactose (●), and OD₆₀₀ in the mixture of both sugars (×).

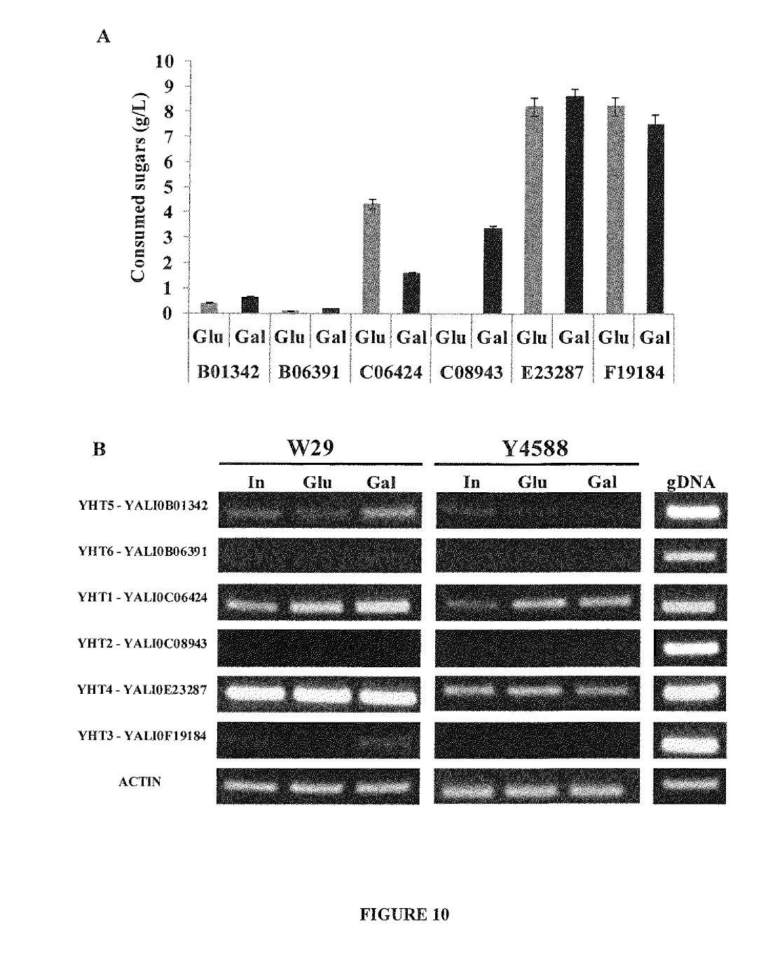

FIG. 10: Sugar consumption by S. cerevisiae null mutants expressing Y. lipolytica hexose transporters after 72 h of growth in YNB medium containing 1% glucose (grey square ▪) or 1% galactose (black square ▪) (A). Expression profiles of Y. lipolytica genes encoding hexose transporters in the W29 and Y4588 strains (B). Yeast were incubated for 3 hours in YNB medium containing 1.0% glucose or 1.0% galactose. The amplification of the PCR fragment in the genomic DNA served as a control for the primers' efficiency. Abbreviation: In—inoculum.

### EXAMPLE: ACTIVATION OF EFFICIENT GALACTOSE UTILIZATION BY YARROWIA LIPOLYTICA

1. Materials and Methods

1.1. Yeast Strains and Plasmids

The strains and plasmids used in this example are listed in Table 1 below.

TABLE 1
Bacteria and yeast strains used in the present example.

| | | |
|---|---|---|
| Name | Relevant genotype | Reference |

E. coli strains

| | | |
|---|---|---|
| JME547 | pUB4-CRE 1 | Fickers et |
| | | al. (2003) |
| JME2110 | JMP62pTEF-scGAL1-URA3ex | This study |
| JME2111 | JMP62pTEF-scGAL1-LEU2ex | This study |
| JME2114 | JMP62pTEF-scGAL7-URA3ex | This study |
| JME2115 | JMP62pTEF-scGAL7-LEU2ex | This study |
| JME2116 | JMP62pTEF-scGAL10-URA3ex | This study |
| JME2117 | JMP62pTEF-scGAL10-LEU2ex | This study |
| JME2542 | JMP62pTEF-ylGAL1-URA3ex | This study |
| JME2543 | JMP62pTEF-ylGAL7-URA3ex | This study |
| JME2544 | JMP62pTEF-ylGAL10E-URA3ex | This study |
| JME2545 | JMP62pTEF-ylGAL10M-URA3ex | This study |
| JME2546 | JMP62pTEF-ylGAL1-LEU2ex | This study |
| JME2547 | JMP62pTEF-ylGAL7-LEU2ex | This study |
| JME2548 | JMP62pTEF-ylGAL10E-LEU2ex | This study |
| JME2549 | JMP62pTEF-ylGAL10M-LEU2ex | This study |
| JME2735 | pRS426URA3-ylGAL1 | This study |
| JME2736 | pRS426URA3-ylGAL7 | This study |
| JME2738 | pRS426URA3-ylGAL10E | This study |
| JME2739 | pRS426URA3-ylGAL10M | This study |

Y. lipolytica strains

| | | |
|---|---|---|
| PO1d | W29 ura3-302 leu2-270 xpr2-322 + pXPR2-SUC2 | Barth and |
| | | Gaillardin |
| | | (1996) |
| W29 | MATa ura3-302 leu2-270 xpr2-322 URA3ex LEU2 | Barth and |
| | | Gaillardin |
| | | (1996) |
| Y3671 | MATa ura3-302 leu2-270 xpr2-322 pTEF-scGAL1-LEU2ex | This study |
| Y3681 | MATa ura3-302 leu2-270 xpr2-322 pTEF-scGAL7-URA3ex | This study |
| Y3682 | MATa ura3-302 leu2-270 xpr2-322 pTEF-scGAL10-URA3ex | This study |
| Y3683 | MATa ura3-302 leu2-270 xpr2-322 pTEF-scGAL1-LEU2ex pTEF-scGAL7-URA3ex | This study |
| Y3684 | MATa ura3-302 leu2-270 xpr2-322 pTEF-scGAL1-LEU2ex pTEF-scGAL10-URA3ex | This study |
| Y3685 | MATa ura3-302 leu2-270 xpr2-322 pTEF-scGAL7-URA3ex pTEF-scGAL10-LEU2ex | This study |
| Y3686 | MATa ura3-302 leu2-270 xpr2-322 pTEF-scGAL1 pTEF-scGAL7 | This study |
| Y3687 | MATa ura3-302 leu2-270 xpr2-322 pTEF-scGAL1 pTEF-scGAL7 pTEF-scGAL10-URA3ex | This study |
| Y4571 | MATa ura3-302 Leu2-270 xpr2-322 pTEF-scGAL1 pTEF-scGAL7 pTEF-scGAL10-URA3ex LEU2 clone1 | This study |
| Y4572 | MATa ura3-302 leu2-270 xpr2-322 pTEF-scGAL1 pTEF-scGAL7 pTEF-scGAL10-URA3ex LEU2 clone5 | This study |
| Y4573 | MATa ura3-302 leu2-270 xpr2-322 pTEF-ylGAL1-URA3ex | This study |
| Y4574 | MATa ura3-302 leu2-270 xpr2-322 pTEF-ylGAL7-URA3ex | This study |
| Y4575 | MATa ura3-302 leu2-270 xpr2-322 pTEF-ylGAL10E-URA3ex | This study |
| Y4576 | MATa ura3-302 leu2-270 xpr2-322 pTEF-ylGAL10M-URA3ex | This study |
| Y4577 | MATa ura3-302 leu2-270 xpr2-322 pTEF-ylGAL1-URA3ex pTEF-ylGAL7-LEU2ex | This study |
| Y4578 | MATa ura3-302 leu2-270 xpr2-322 pTEF-ylGAL1-URA3ex pTEF-ylGAL10E-LEU2ex | This study |
| Y4579 | MATa ura3-302 leu2-270 xpr2-322 pTEF-ylGAL1-URA3ex pTEF-ylGAL10M-LEU2ex | This study |
| Y4580 | MATa ura3-302 leu2-270 xpr2-322 pTEF-ylGAL7-URA3ex pTEF-ylGAL10E-LEU2ex | This study |
| Y4581 | MATa ura3-302 leu2-270 xpr2-322 pTEF-ylGAL7-URA3ex pTEF-ylGAL10M-LEU2ex | This study |
| Y4582 | MATa ura3-302 leu2-270 xpr2-322 pTEF-ylGAL10a-URA3ex pTEF-ylGAL10M-LEU2ex | This study |
| Y4583 | MATa ura3-302 leu2-270 xpr2-322 pTEF-ylGAL1 pTEF-ylGAL7 | This study |
| Y4584 | MATa ura3-302 leu2-270 xpr2-322 pTEF-ylGAL1 pTEF-ylGAL10E | This study |
| Y4585 | MATa ura3-302 leu2-270 xpr2-322 pTEF-ylGAL1 pTEF-ylGAL7 pTEF-ylGALl10E-LEU2ex | This study |
| Y4586 | MATa ura3-302 leu2-270 xpr2-322 pTEF-ylGAL1 pTEF-ylGAL7 pTEF-ylGAL10M-LEU2ex | This study |
| Y4587 | MATa ura3-302 leu2-270 xpr2-322 pTEF-ylGAL1 pTEF-ylGAL10a pTEF-ylGAL10M-LEU2ex | This study |
| Y4588 | MATa ura3-302 leu2-270 xpr2-322 pTEF-ylGAL1 pTEF-ylGAL7 pTEF-ylGALl10E-LEU2ex pTEF-ylGAL10M-URA3ex | This study |
| Y4629 | MATa ura3-302 leu2-270 xpr2-322 pTEF-ylGAL1-URA3ex LEU2 | This study |
| Y4630 | MATa ura3-302 leu2-270 xpr2-322 pTEF-ylGAL7-URA3ex LEU2 | This study |
| Y4631 | MATa ura3-302 leu2-270 xpr2-322 pTEF-ylGAL10E-URA3ex LEU2 | This study |
| Y4632 | MATa ura3-302 leu2-270 xpr2-322 pTEF-ylGAL10M-URA3ex LEU2 | This study |
| Y4633 | MATa ura3-302 leu2-270 xpr2-322 pTEF-ylGAL1 pTEF-ylGAL7 pTEF-ylGALl10E-LEU2ex URA3ex | This study |
| Y4634 | MATa ura3-302 leu2-270 xpr2-322 pTEF-ylGAL1 pTEF-ylGAL7 pTEF-ylGAL10M-LEU2ex URA3ex | This study |
| Y4635 | MATa ura3-302 leu2-270 xpr2-322 pTEF-ylGAL1 pTEF-ylGAL10a pTEF-ylGAL10M-LEU2ex URA3ex | This study |

S. cerevisiae strains

| | | |
|---|---|---|
| Y4473 | Mat a; his3Δ1; leu2Δ0; met15Δ0; ura3Δ0; YBR018c::kanMX4 (ΔGal7) | Euroscarf |
| | | (Y13155) |
| Y4474 | Mat a; his3Δ1; leu2Δ0; lys2Δ0; ura3Δ0; YBR019::kanMX4 (ΔGal10) | Euroscarf |
| | | (Y13156) |
| Y4475 | Mat a; his3Δ1; leu2Δ0; lys2Δ0; ura3Δ0; YBR020w::kanMX4 (ΔGal1) | Euroscarf |
| | | (Y13157) |
| Y4846 | Mat a; his3Δ1; leu2Δ0; lys2Δ0; ura3Δ0; YDR009w::kanMX4 (ΔGal3) | Euroscarf |
| | | (Y13949) |
| Y4591 | Mat a; his3Δ1; leu2Δ0; lys2Δ0; ura3Δ0; YBR020w::kanMX4 URA3-EV(pRS426TEF) | This study |
| Y4592 | Mat a; his3Δ1; leu2Δ0; lys2Δ0; ura3Δ0; YPL248c::kanMX4 URA3-EV(pRS426TEF) | This study |
| Y4593 | Mat a; his3Δ1; leu2Δ0; met15Δ0; ura3Δ0; YBR018c::kanMX4 URA3-EV(pRS426TEF) | This study |
| Y4594 | Mat a; his3Δ1; leu2Δ0; lys2Δ0; ura3Δ0; YBR019c::kanMX4 URA3-EV(pRS426TEF) | This study |
| Y4595 | Mat a; his3Δ1; leu2Δ0; lys2Δ0; ura3Δ0; YBR020w::kanMX4 pTEF-ylGAL1-URA3 | This study |
| Y4596 | Mat a; his3Δ1; leu2Δ0; met15Δ0; ura3Δ0; YBR018c::kanMX4 pTEF-ylGAL7-URA3 | This study |
| Y4597 | Mat a; his3Δ1; leu2Δ0; lys2Δ0; ura3Δ0; YBR019c::kanMX4 pTEF-ylGAL10E-URA3 | This study |
| Y4690 | Mat a; his3Δ1; leu2Δ0; lys2Δ0; ura3Δ0; YBR019c::kanMX4 pTEF-ylGAL10M-URA3 | This study |
| Y4874 | Mat a; his3Δ1; leu2Δ0; lys2Δ0; ura3Δ0; YDR009w::kanMX4 URA3-EV(pRS426TEF) | This study |
| Y4875 | Mat a; his3Δ1; leu2Δ0; lys2Δ0; ura3Δ0; YDR009w::kanMX4 URA3-ylGAL1(ΔGal3) | This study |
| B01342 | leu2-3,112 ura3-52 trp1-289 his31 MAL2-8C SUC2 hxt17 hxt13 hxt15 hxt16 hxt14 hxt12 hxt9 hxt11 hxt10 hxt8 hxt5-1-4 | Lazar et al., |
| | hxt2 hxt367 gal2 slt1 agt1 ydl247 yjr160c URA3-YALI0B01342 | in preparation |
| B06391 | leu2-3,112 ura3-52 trp1-289 his31 MAL2-8C SUC2 hxt17 hxt13 hxt15 hxt16 hxt14 hxt12 hxt9 hxt11 hxt10 hxt8 hxt5-1-4 | Lazar et al., |
| | hxt2 hxt367 gal2 slt1 agt1 ydl247 yjr160c URA3-YALI0B06391 | in preparation |
| C06424 | leu2-3,112 ura3-52 trp1-289 his31 MAL2-8C SUC2 hxt17 hxt13 hxt15 hxt16 hxt14 hxt12 hxt9 hxt11 hxt10 hxt8 hxt5-1-4 | Lazar et al., |
| (YHT1) | hxt2 hxt367 gal2 slt1 agt1 ydl247 yjr160c URA3-YALI0C06424 | in preparation |
| C08943 | leu2-3,112 ura3-52 trp1-289 his31 MAL2-8C SUC2 hxt17 hxt13 hxt15 hxt16 hxt14 hxt12 hxt9 hxt11 hxt10 hxt8 hxt5-1-4 | Lazar et al., |
| (YHT2) | hxt2 hxt367 gal2 slt1 agt1 ydl247 yjr160c URA3-YALI0C08943 | in preparation |
| E23287 | leu2-3,112 ura3-52 trp1-289 his31 MAL2-8C SUC2 hxt17 hxt13 hxt15 hxt16 hxt14 hxt12 hxt9 hxt11 hxt10 hxt8 hxt5-1-4 | Lazar et al., |
| (YHT4) | hxt2 hxt367 gal2 slt1 agt1 ydl247 yjr160c URA3-YALI0E23287 | in preparation |
| F19184 | leu2-3,112 ura3-52 trp1-289 his31 MAL2-8C SUC2 hxt17 hxt13 hxt15 hxt16 hxt14 hxt12 hxt9 hxt11 hxt10 hxt8 hxt5-1-4 | Lazar et al., |
| (YHT3) | hxt2 hxt367 gal2 slt1 agt1 ydl247 yjr160c URA3-YALI0F19184 | in preparation |

All the Y. lipolytica transformants were created by employing the genetic background of the W29 wild-type strain (Barth and Gaillardin, 1996). The auxotrophic strain PO1d (Ura“Leu”) was derived from W29 and was also used in this study (Barth and Gaillardin 1996). Because many different strains overexpressing Y. lipolytica GAL genes were created, only the quadruple overexpressing strain, Y4588, which overexpressed all four of the ylGAL genes, is described in detail here. Construction of the other strains is depicted in FIG. 1. Plasmids containing ylGAL genes were constructed using URA3ex and LEU2ex selection markers. Strain Y4573, which overexpressed the Y. lipolytica ylGAL1 gene (YALI0C13090g), was obtained by introducing the overexpression cassette from JME2542 containing the URA3ex selection marker into PO1d. Subsequently, the cassette containing the ylGAL7 gene (YALI0F23947g) and a LEU2ex marker taken from JME2547 was introduced into the Y4573 strain, thus generating the prototrophic Y4577 strain. To excise both selection markers, Y4577 was transformed with JME547 plasmid containing Cre-Lox recombinase and hygromycin selection. This process generated the auxotrophic Y4583 strain (Ura“Leu”). Immediately afterwards, the ylGAL10E gene (YALI0E26829g) overexpression cassette containing the LEU2ex marker from a JME2548 plasmid was introduced into the Y4583. Then, the cassette containing the ylGAL10M gene (YALI0C09570g) and the URA3ex marker taken from a JME2545 plasmid were introduced, generating Y4588, which overexpressed the entire Leloir pathway.

Y. lipolytica strains expressing S. cerevisiae GAL genes were constructed as depicted in FIG. 2. All the plasmids were prepared as described above.

Functional complementation analysis of the Y. lipolytica GAL genes was performed using S. cerevisiae strains from which the GAL genes had been deleted. The Y. lipolytica galactokinase (ylGAL1) gene was introduced into S. cerevisiae Δgal1 (Y4475) as well as into the Δgal3 (Y4846) strains using JME2735 plasmid, thus generating the Y4595 and Y4875 strains, respectively. To complement the Δgal7 strain, a JME2736 plasmid (ylGAL7) was introduced into Y4473, generating Y4596. Finally, Δgal10 was complemented separately by the ylGAL10E and ylGAL10M genes, which was accomplished by introducing JME2738 and JME2739 plasmids into the Y4474 strain, resulting in the creation of Y4597 and Y4690, respectively. All the S. cerevisiae deletion mutants—the control strains—were also complemented for uracil auxotrophy by the insertion of an empty pRS426TEF vector.

All the genes introduced into Y. lipolytica and S. cerevisiae were under the control of the constitutive TEF promoter (Mumberg et al., 1995; Müller et al., 1998). Transformation of Y. lipolytica and S. cerevisiae was performed using the lithium acetate procedure (Xuan et al., 1990). Expression cassettes were integrated into the genome of Y. lipolytica (Mauersberger et al., 2001) or were in the replicative pRS426TEF vector in S. cerevisiae (Mumberg et al., 1995).

1.2. General Genetic Techniques and Plasmid Construction

Standard molecular genetics techniques were used throughout this study (as per Sambrok and Russell, 2001). Restriction enzymes were obtained from New England Biolabs (Ipswich, England). Genomic DNA from the transformants was prepared as described in Querol et al. (1992). PCR amplification was performed using an Eppendorf 2720 thermal cycler; both Taq (Promega, Madison, Wis.) and Pfu (Stratagene, La Jolla, Calif.) DNA polymerases were employed. PCR fragments were then purified with a QIAgen Purification Kit (Qiagen, Hilden, Germany), and DNA fragments were recovered from the agarose gels using a QIAquick Gel Extraction Kit (Qiagen, Hilden, Germany). The Staden software package was used for gene sequence analysis (Dear and Staden, 1991). The GAL genes were amplified using the primers listed in Table 2 below.

TABLE 2
List of primers used in the present example.

| | | | | SEQ |
|---|---|---|---|---|
| | | Restriction | Amplified | ID |
| Primer | Sequence | site | gene | NO: |
| Gal1-BamHI-fwd | gcgcggatccatgactaaatctcattcagaagaagtgattg | BamHI | scGAL1 | 30 |
| Gal1-AvrII-rev | gcgccctaggttataattcatatagacagctgcccaatgc | AvrII | | 31 |
| ScGal7-BamHI-fwd | gcgcggatccatgactgctgaagaatttg | BamHI | scGAL7 | 32 |
| Gal7-AvrII-rev | gcgccctaggttacagtattgtagataatgaatc | AvrIl | | 33 |
| Gal10-BamHI-fwd | gcgcggatccatgacagctcagttacaaag | BamHI | scGAL10 | 34 |
| Gal10-AvrII-rev | gcgccctaggtcaggaaaatctgtagacaatc | AvrII | | 35 |
| YLGal1Short-BamHI-fwd | gtgaggatccatgtctatcagcaccctgcc | BamHI | ylGAL1 | 36 |
| YlGal1-AvrII-rev | gcgccctaggttacaaatcaacaatagcacatccc | AvrII | | 37 |
| YlGal7-BamHI-fwd | gagaggatccatgactcttgtggcatccg | BamHI | ylGAL7 | 38 |
| YlGal7-AvrII-rev | gcgccctaggttacaaatcatacaaccgcttgg | AvrII | | 39 |
| YlGal10E-BglII-fwd | gagaagatctatgacccaggagctgctgaac | BglII | ylGAL10E | 40 |
| YlGal10E-SpeI-rev | gcgcactagtttacttgtcatgacggtcattttgtaacc | SpeI | | 41 |
| YlGal10M-BamHI-fwd | gagaggatccatgaccttttgcaacacgggagcaac | BamHI | ylGAL10M | 42 |
| YlGal10M-AvrII-rev | gcgccctaggtcactcccgctccaaaacatagataatcttagc | AvrII | | 43 |
| YLGal1Short-SpeI-fwd | gcgcactagtatgtctatcagcaccctgcc | SpeI | ylGAL1 | 44 |
| YlGal1-SmaI-rev | gcgccccgggttacaaatcaacaatagcacatccctc | SmaI | | 45 |
| YlGa17-SpeI-fwd | gagaactagtatgactcttgtggcatccg | SpeI | ylGAL7 | 46 |
| YlGa17-SmaI-rev | gcgacccgggttacaaatcatacaaccgcttggc | SmaI | | 47 |
| YlGa110E-SpeI-fwd | gagaactagtatgacccaggctgctgag | SpeI | ylGAL10E | 48 |
| YlGall0E-SmaI-rev | gagaccccggttacttgtcatgacggtcctttttg | SmaI | | 49 |
| YLGall0M-SpeI-fwd | gcgcactagtatgaccttttgcaacacgggagcaac | SpeI | ylGAL10M | 50 |
| YLGal10M-EcoRI-rev | gcgcgaattctcactcccgctccaaaacatagataatcttag | EcoRI | | 51 |

Primers used for RT-PCR and qRT-PCR

| | | | |
|---|---|---|---|
| YlGal1-RT-F | tcttcaactgctcctgtcct | ylGAL1 | 52 |
| YlGal1-RT-R | ctatcgtaatcgacaccatcc | | 53 |
| Y1Gal7-RT-F | ccatcaaaaagagaccaag | ylGAL7 | 54 |
| YlGal7-RT-R | atcgcagaagaggagggtag | | 55 |
| YlGal10E-RT-F | tggtgacgactacccctct | ylGAL10E | 56 |
| YlGal10E-RT-R | cggttcctaggttccattct | | 57 |
| YlGal10M-RT-F | tccatctccctcaatggaac | ylGAL10M | 58 |
| YlGal10M-RT-R | gccggtatagacctggaaac | | 59 |

The restriction sites in the primer sequences enabled the genes to be cloned into JME1128 plasmids that had been digested with BamHI-AvrII, as previously described elsewhere (Beopoulos et al., 2008; Dulermo et al., 2013). Auxotrophies were restored via excision using the Cre-lox recombinase system following transformation with the replicative plasmid pUB4-Cre1 (JME547) (Fickers et al., 2003).

To complement the S. cerevisiae strains whose GAL genes had been deleted, the corresponding genes were cloned in a pRS426 vector using the constitutive TEF promoter and the uracil selection marker. The corresponding ylGAL genes were amplified and digested, as described above, with the restriction enzymes listed in Table 2 above.

1.3. Growth Media

Media and growth conditions for Escherichia coli were identical to those as described by Sambrook and Russell (2001), as were those of Y. lipolytica (Barth and Gaillardin, 1996). Rich (YPD) medium was prepared using 20 g·L⁻¹ Bacto™ Peptone (Difco, Paris, France), 10 g·L⁻¹ yeast extract (Difco), and 20 g·L⁻¹ glucose (Merck, Fontenay-sous-Bois, France). Minimal (YNB) medium was prepared using 1.7 g·L⁻¹ yeast nitrogen base (without amino acids and ammonium sulphate, Difco), 10 g·L⁻¹ glucose (Merck), 5 g·L⁻¹ NH₄Cl, and 50 mM phosphate buffer (pH 6.8). To complement the auxotrophies, 0.1 g·L⁻¹ uracil or leucine (Difco, Paris, France) were added as necessary.

However, the YNB media on which they were grown contained the following: 10.0 g·L⁻¹ of either glucose or galactose; 6.5 g·L⁻¹ of Yeast Nitrogen Base (without amino acids and ammonium sulphate, Difco); 10.0 g·L⁻¹ of (NH₄)₂SO₄; 0.018 g·L⁻¹ of leucin; 0.0115 g·L⁻¹ of histidin; and 0.025 g·L⁻¹ of lysin.

1.4. Growth in Microtiter Plates

Precultures were obtained from frozen stock, inoculated into tubes containing 5 mL YPD medium, and cultured overnight (170 rpm, 28° C.). They were then washed with sterile distilled water; cell suspensions were adjusted to an OD₆₀₀ of 0.1. Yeast strains were grown in 96-well plates in 200 μl of minimal YNB medium containing 10 g·L⁻¹ of either glucose or galactose. Culturing was repeated three times; 2-3 technical replicates were performed for each condition. Cultures were maintained at 28° C. under constant agitation using a Biotek Synergy MX microtiter plate reader (Biotek Instruments, Colmar, France); each culture's optical density at 600 nm was measured every 20 min for 72 h.

1.5. Growth Conditions in the Experiments

Initial precultures were established by inoculating 50 mL of YPD medium in 250 mL Erlenmeyer flasks with the yeast strains; this was followed by an overnight shaking step at 28° C. and 170 rpm. The cell suspensions were washed three times with sterile distilled water and used to inoculate 50 mL of YNB medium to an OD₆₀₀ of 0.25. The cultures were grown until all the available sugar had been consumed.

In the galactose utilization experiment, the YNB medium contained one of the following: 1.0 g·L⁻¹ of glucose; 1.0 g·L⁻¹ of galactose; 1.0 g·L⁻¹ of both glucose and galactose; or 10.0 g·L⁻¹ of both glucose and galactose. Additionally, galactose utilization was observed in YNB medium containing 10.0 g·L⁻¹ of galactose and either 1.0; 2.0; 4.0; 6.0; 8.0; or 10.0 g·L⁻¹ of glucose.

1.6. Analysis of Growth on Galactose by Drop Test

To analyze the different strains' growth on galactose, a drop test was performed on cultures grown on YNB plates. The Y. lipolytica strains were grown in 5 mL of YPD medium for 24 h. The cell suspensions were then washed twice with water and re-suspended at an OD₆₀₀ of 1. Successive 10-fold dilutions were performed (10⁰-10⁻⁵), and 5 μl of each dilution were spotted onto YNB plates containing 10.0 g·L⁻¹ of glucose, 1.0 g·L⁻¹ of galactose, or 10.0 g·L⁻¹ of galactose. Pictures were taken after the cultures had been incubated at 28° C. for 48 h. The same protocol was used for the S. cerevisiae strains. However, the YNB media on which they were grown contained the following: 10.0 g·L⁻¹ of either glucose or galactose; 6.5 g·L⁻¹ of Yeast Nitrogen Base (without amino acids and ammonium sulphate, Difco); 10.0 g·L⁻¹ of (NH₄)₂SO₄; 0.018 g·L⁻¹ of leucin; 0.0115 g·L⁻¹ of histidin; and 0.025 g·L⁻¹ of lysin.

1. 7. Analysis of Galactose Uptake by Y. lipolytica Hexose Transporters

To analyze galactose uptake, Y. lipolytica hexose transporters were expressed one at a time in the S. cerevisiae hxt null mutant strain EBY.VW4000 (kindly provided by E. Boles, Goethe University, Frankfurt am Main, Germany) using a replicative pRS426 vector containing the TEF promoter. The cells were grown in 5 mL of YNB medium composed of 6.5 g·L⁻¹ of Yeast Nitrogen Base (without amino acids and ammonium sulphaₜe, Difco); 10.0 g·L⁻¹ of (NH4)₂SO₄ as well as leucin, histidin and tryptophan to complement auxotrophies and containing 20 g·L⁻¹ of maltose; the medium was refreshed 3 times per 24 h period to increase 2μ plasmid copy number. The cell suspensions were washed three times with sterile distilled water and used to inoculate 50 mL of YNB medium that contained either 10.0 g·L⁻¹ of glucose or 10.0 g·L⁻¹ of galactose. The culture conditions were as described above (paragraph 1.5). OD₆₀₀ and sugar concentration were analyzed.

1.8. Conditions and Media Used for Lipid Biosynthesis from Galactose in Flasks and Bioreactors

The precultures were prepared as described above (1.5). The main culture was grown on 50 mL of YNB medium (C/N 60) containing the following: galactose 60.0 g; YNB 1.7 g; NH₄Cl 1.5 g; 0.7 g KH₂PO₄, and 1.0 g MgSO₄×7H₂O in 1 L. The pH was kept at 6.8 using 0.05 M phosphate buffer. Tap water was used as a source of microelements. To increase the C/N ratio of the medium to 100, 1.25 g·L⁻¹ of NH₄Cl was used. Culture conditions were as described above (2.5.1).

Lipid biosynthesis was also evaluated using batch cultures (BC) that were kept in 5-L stirred-tank BIO-STAT B-PLUS bioreactors (Sartorius, Frankfurt, Germany) for 96 h under the following conditions: 2-L working volume, 28° C., 800 rpm of agitation, and 3.5-L min⁻¹ aeration rate. The production medium was prepared as described above. The pH was kept at 6.8 using a 40% (w/v) NaOH solution. The cultures were grown in 0.2 L of YPD medium in 0.5-L flasks at 170 rpm, at 28° C. for 48 h. The volume of the inocula added to the bioreactor cultures was equal to 10% of the total working volume.

1.9. Analysis of ylGAL Gene and Hexose Transporter Expression

For the expression experiments, the wild-type strain (W29) and the quadruple mutant (Y4588) were grown in YNB medium that had been supplemented with 10 g·L⁻¹ glucose; they were kept at 28° C. for 16 h. Immediately afterwards, the cell suspensions were washed twice with distilled water and transferred into fresh YNB medium that contained either 1.0 or 10.0 g·L⁻¹ of glucose, galactose, or mannose. Samples were harvested at 3 h post inoculation; three replicates were obtained. For the kinetics experiments, the same protocol was followed, except that samples were harvested at 3, 6, 9, and 24 h post inoculation. All the samples were frozen in liquid nitrogen and stored at −80° C.

Total RNA was extracted using the RNeasy Mini Kit (Qiagen, Hilden, Germany), and 1.5 μg of each sample was treated with DNase (Ambion, Life Technologies). cDNA was synthesized using the Maxima First Strand cDNA Synthesis Kit for RT-qPCR (Thermo Scientific). PCR reactions were performed using specific primers that targeted the 3′ end of the genes (Table 2). For the semi-quantitative RT-PCR analyses, PCR was performed using the GoTaq DNA Polymerase Kit (Promega). For the quantitative RT-PCR analyses, gDNA dilutions were first tested for primer efficiency and then retained if their efficiency rating was higher than 90%. Amplifications were carried out using the SsoAdvanced Universal SYBR Green Supermix Kit (BIO-RAD). The following program was used: 98° C. for 3 min, followed by 40 cycles of 98° C. for 15 sec, 58° C. for 30 sec, and 72° C. for 30 sec. Finally, melting curves were generated to confirm amplification specificity. Both ΔCT and ΔΔCT methods were used to calculate relative expression levels; a constitutive gene, actin, was utilized as the reference control (Schmittgen and Livak, 2008).

1.10. Analytical Techniques Used in this Study

Lipid bodies were stained with BodiPy® Lipid Probe (2.5 mg·mL⁻¹ in ethanol; Invitrogen). Cell suspension samples (OD₆₀₀=5) were incubated for 10 min at room temperature. Images were obtained using a Zeiss Axio Imager M2 microscope (Zeiss, Le Pecq, France) equipped with a 100× objective lens and Zeiss filter sets 45 and 46 for fluorescence microscopy. Axiovision 4.8 software (Zeiss, Le Pecq, France) was used for image acquisition.

The fatty acids occurring in 15-mg aliquots of freeze-dried cells were converted into methyl esters using the method described in Browse et al. (1986). They were then analyzed using a gas chromatograph (GC). GC analysis of the methyl esters was performed using a Varian 3900 instrument equipped with a flame ionization detector and a Varian FactorFour vf-23ms column, for which bleed specification at 260° C. was 3 pA (30 m, 0.25 mm, 0.25 μm). Fatty acids were identified by comparing the GC patterns of their methyl esters to those of commercial methyl ester standards (FAME32; Supelco); the amounts present were quantified using the internal standard method, which involved the addition of 50 μg of commercial C17:0 (Sigma).

Citric acid, glucose, fructose, and sucrose were identified and quantified by HPLC (UltiMate 3000, Dionex-Thermo Fisher Scientific, UK) using an Aminex HPX87H column coupled with UV (210 nm) and RI detectors. The column was eluted with 0.01 N H₂SO₄ at a flow rate of 0.6 mL·min⁻¹ at room temperature. Compounds were identified and quantified via comparisons to standards. Before undergoing HPLC analysis, the samples were filtered on membranes with a pore size of 0.45 μm.

To determine dry biomass, cell pellets from the 15-mL culture samples were washed twice with distilled water, filtered on the above membranes, and dried at 105° C. using a WPS 110S weight dryer (Radwag, Poznań, Poland) until a constant mass was reached.

2. Results

2.1. Y. lipolytica Has All the Structural Leloir Pathway Genes

Although Y. lipolytica does not normally grow on galactose, its genome contains all the genes of the Leloir pathway (Slot and Rokas, 2010).

In Y. lipolytica, UDP-glucose-4-epimerase and galactose mutarotase are encoded by two different genes, which gave rise to the following four structural genes: ylGAL1 (YALI0C13090g, galactokinase), ylGAL7 (YALI0F23947g, galactose-1-phosphate uridyl transferase), ylGAL10E (YALI0E26829g, UDP-glucose-4 Epimerase), and ylGAL10M (YALI0C09570g, galactose Mutarotase) (Table 3).

TABLE 3
Comparison of GAL genes among Y. lipolytica, S. cerevisiae, and H. jecorina.

| | Y. lipolytica | | | S. cerevisiae | | | |
|---|---|---|---|---|---|---|---|
| | | | Protein | | | Protein | |
| | | | length | | | length | Identity |
| Enzyme name | Abbreviation | Gene | (aa) | Abbreviation | Gene | (aa) | (%) |
| Galactokinase | ylGAL1 | YALI0C13090g | 476 | scGAL1 | YBR020w | 528 | 40 |
| Galactose-1- | ylGAL7 | YALI0F23947g | 352 | scGAL7 | YBR018c | 366 | 49 |
| phosphate | | | | | | | |
| uridyl | | | | | | | |
| transferase | | | | | | | |
| UDP-glucose-4- | ylGAL10E | YALI0E26829g | 369 | scGAL10* | YBR019c | 699 | 62 |
| epimerase | | | | | | | |
| Galactose | ylGAL10M | YALI0C09570g | 327 | | | | 26 |
| mutarotase | | | | | | | |

| | | Hypocrea jecorina | | | | |
|---|---|---|---|---|---|---|
| | | | | Protein | | |
| | Similarity | | | length | Identity | Similarity |
| Enzyme name | (%) | Abbreviation | Locus | (aa) | (%) | (%) |
| Galactokinase | 55 | Gal1 | XP_006961307 | 526 | 40 | 56 |
| Galactose-1- | 63 | Gal7 | XP_006968597 | 382 | 49 | 61 |
| phosphate | | | | | | |
| uridyl | | | | | | |
| transferase | | | | | | |
| UDP-glucose-4- | 76 | Gal10E | AF439323_1 | 370 | 59 | 69 |
| epimerase | | | | | | |
| Galactose | 42 | Gal10M | XP_006964994 | 342 | 29 | 36 |
| mutarotase | | | | | | |

*in S. cerevisiae, the GAL10 gene contains the two domains epimerase and mutarotase.

Although all the structural genes necessary for galactose utilization are present in Y. lipolytica's genome, the genes known to encode regulatory proteins in S. cerevisiae (i.e., scGAL3, scGAL4, and scGAL80) are not. This genetic difference accounts for the differences observed between the two species in the galactose utilization pathway. In addition, GAL4 binding sites are not present in the promoter regions of ylGAL genes (data not shown), which is also the case for H. jecorina (Seiboth et al., 2002). Despite this fact, H. jecorina is able to grow well on galactose.

2.2 Y. lipolytica GAL Genes Are Expressed

To examine ylGAL gene expression, Y. lipolytica was grown on a suite of media containing two different concentrations (0.1% and 1.0%) of three different monosaccharides (galactose, glucose, and mannose), and transcription analyses were performed (FIG. 3A). Galactose was the target compound, glucose was a potential repressor, and mannose was a neutral sugar in catabolite repression. The three main ylGAL genes (ylGAL1, ylGAL7, ylGAL10E) were expressed under all of the conditions, but ylGAL10M, the least important gene in galactose utilization, showed very weak expression (FIG. 3A).

Since the ylGAL genes were not repressed by glucose, a kinetics analysis of their quantitative expression on glucose and galactose was conducted (FIGS. 3B, C). The gene profiles show that, on glucose, expression of the UDP-glucose-4-epimerase gene (ylGAL10E) was constant, whereas expression of galactokinase (ylGAL1) and galactose-1-phosphate uridyl transferase (ylGAL7) genes increased over time (FIG. 3B). Once again, the galactose mutarotase gene (ylGAL10M) had the lowest expression levels. On galactose, there was a slight increase in expression over time: it reached between 2.7 and 4.6 after 24 h (FIG. 3C). Therefore, the ylGAL genes were upregulated in the presence of galactose.

2.3 Y. lipolytica GAL Genes Are Functional

To investigate the activity of the proteins encoded by the Y. lipolytica GAL genes, a functional complementation test was performed using S. cerevisiae. The ylGAL genes were amplified and cloned into a multicopy pRS426 vector under the control of the scTEF promoter. The S. cerevisiae transformants obtained were subsequently tested to determine if they could grow when galactose was the sole carbon source (FIG. 4). The three cloned ylGAL genes, ylGAL1, ylGAL7, and ylGAL10E, complemented their corresponding S. cerevisiae GAL-deletion mutants and restored growth on galactose. This result shows that the ylGAL genes encode functional Leloir proteins. In addition, the ylGAL1p gene, which complemented the S. cerevisiae Δgal1 mutant, was not able to restore normal growth on galactose for the S. cerevisiae Δgal3 mutant (FIG. 4).

2.4 Validation of Leloir Pathway Functionality in Y. lipolytica

Galactose utilization by Y. lipolytica was investigated. Results are shown in FIG. 5. The ylGAL genes were expressed when the yeast was grown on glucose, but galactose alone did not allow growth. As a result, growth and sugar consumption by Y. lipolytica W29 (the wild-type strain) in YNB medium containing a mixture of glucose and galactose (0.1% or 1.0%) were analyzed. First, a control experiment was conducted in which growth and sugar consumption on glucose versus galactose were examined. As expected, when galactose was the sole carbon source, it was not consumed, whereas 0.1% and 1.0% glucose were depleted within 12 and 32 h, respectively (FIGS. 5A, C). Surprisingly, when the same strain was grown in medium containing both 0.1% glucose and 0.1% galactose, it was able to use both sugars (FIG. 5B). The glucose utilization rate was the same as for the glucose-only culture (0.083 g·L⁻¹·h⁻¹). In contrast, galactose was initially used at a very low rate (0.009 g·L⁻¹·h⁻¹); however, after the glucose was completely consumed, the galactose utilization rate increased (0.032 g·L⁻¹·h⁻¹). Additionally, sugar consumption was also tested using cultures containing higher concentrations of both monosaccharides (1%) to eliminate the influence of any putative sugar sensors, which could change carbon metabolism (FIG. 5D). As in the previous experiment, Y. lipolytica W29 used both glucose and galactose from the beginning, but the glucose consumption rate was much higher than the galactose consumption rate (0.313 g·L⁻¹·h⁻¹ versus 0.083 g·L⁻¹·h⁻¹, respectively). Once all the glucose had been consumed, the galactose utilization rate climbed significantly (0.250 g·L⁻¹·h⁻¹). It is worth noting that, not only was galactose consumed by the cells, but it was also partially used for biomass production (FIGS. 5B, D).

The concentration of glucose that Y. lipolytica requires to be able to use galactose was determined. Y. lipolytica W29 was therefore grown in a suite of media containing 1.0% galactose and a range of glucose, from 0.1 to 1.0%. At a glucose concentration of 0.1%, growth was very weak and galactose was not consumed (FIG. 6A). At higher glucose concentrations (0.2-0.4%), a small amount of galactose was also utilized; however, after the glucose was used up, galactose consumption stopped (FIGS. 6B, C). At even higher glucose concentrations, Y. lipolytica consumed galactose more efficiently, following a short delay (FIGS. 6D-F). Galactose consumption increased once the glucose had been drained from the medium, which is also when the cells enter into the stationary phase (FIGS. 6D-F).

2.5. Overexpression of All Y. lipolytica GAL Genes Induces Efficient Growth on Galactose

All the ylGAL genes were placed under the control of the strong constitutive TEF promoter and introduced individually or in various combinations into the Y. lipolytica PO1d background. The resulting strains were then tested to see if they could grow on galactose alone. When the transformants were grown on glucose, they showed no morphological changes, suggesting that their physiology was not affected (FIG. 7A). Mutants in which ylGAL1, ylGAL7, and ylGAL10E were simultaneously overexpressed could grow on 0.1% and 1.0% galactose (FIG. 7A, line 12). Mutarotase activity, which is not required for galactose metabolism, nonetheless appeared to improve galactose utilization (FIG. 7A, line 15). Interestingly, the overexpression of all the S. cerevisiae Leloir genes in Y. lipolytica resulted in weak, delayed growth (FIG. 8A). The improper folding of scGAL10 may have been responsible; its counterpart in Y. lipolytica exists as two proteins. Additional analysis of growth patterns showed that the Y. lipolytica transformants, with the exception of Y4585 and Y4588, took a long time to adapt to using galactose. The Y4577 strain, in which ylGAL1 and ylGAL7 were overexpressed, began to grow after two weeks of incubation but revealed normal morphology on plates (FIG. 8B). Additionally, the Y4578 strain, in which ylGAL1 and ylGAL10E were overexpressed, also began to grow after two weeks of incubation, albeit much less efficiently than Y4577 (FIG. 8B). This finding underscores that ylGAL7 expression results in functional protein biosynthesis but not normal levels for galactose utilization.

The strong increase in gene expression in the quadruple mutant (Y4588) showed that, when galactose concentrations were low, each gene was overexpressed compared to the parental strain; for instance, expression was 400 times higher for ylGAL10M (FIG. 7B). Surprisingly, when galactose concentrations were high (1.0%), the change in gene expression levels was not so sharp, especially in the case of ylGAL10E. The parental strain expresses ylGAL10E at high levels when grown in 1% galactose (FIG. 7B). Overexpressing all four of the ylGAL genes efficiently activated the Leloir pathway and created a strain that can use galactose as its sole carbon source.

2.6. Overexpression of ylGAL Genes Changed Sugar Utilization Kinetics in Y. lipolytica Without Changing Transporters Expression

Glucose must be present in the culture medium for the Y. lipolytica wild type (W29) strain to be able to utilize galactose. Overexpression of all the genes in the Leloir pathway allowed Y. lipolytica cells to grow on media whose sole carbon source was galactose. It was then determined if glucose had an effect on galactose utilization in the Y. lipolytica quadruple mutant (Y4588). To this end, its growth and sugar utilization in 1% glucose, 1% galactose, and a mixture of 1% glucose and 1% galactose were characterized. The kinetics of sugar consumption were the same when the strain was grown in the single sugar cultures, reaching 0.48-49 g·L⁻¹·h⁻¹; slightly more biomass was produced when the strain was grown on glucose alone (FIG. 9A). However, sugar utilization dynamics changed in the mixed medium (FIG. 9B). The galactose utilization rate maxed out at 0.11 g·L⁻¹·h⁻¹, while the glucose utilization rate declined to 0.39 g·L⁻¹·h⁻¹. This same pattern was also seen at lower concentrations of both sugars (0.1% and 0.5%; data not shown). After the lag phase, galactose utilization remained slow and constant, while glucose consumption decreased significantly when the cells reached the stationary phase (FIG. 9B). Overall, growth and sugar consumption were the same on the glucose-only and mixed-sugar media; for instance, at 18 h of incubation, consumption was around 5.5-6 g·L⁻¹ for both (FIG. 9B). Even though the galactose utilization pathway had clearly been activated, glucose was obviously the preferred carbon source.

The transport of galactose inside the cell is the first step in the metabolic process. It may be modified in the quadruple mutant or may be affected by the presence of glucose. In Y. lipolytica, six genes have been identified as coding hexose transporters from a HXT-like family; they have been named in series, YHT1 to YHT6, and have been reported to transport of galactose (Lazar et al., in preparation; Young et al., 2011). The uptake efficiency of both galactose and glucose was compared in S. cerevisiae hxt null mutants transformed with each of these six genes (FIG. 10A). Four of the transporters, Yht1-Yht4, allowed significant galactose or glucose consumption; uptake efficiency, however, varied (FIG. 10A). Only two of these transporter genes, YHT1 and YHT4, were expressed at similarly high levels in Y. lipolytica W29 and Y4588 strains grown in either 1% glucose or 1% galactose (FIG. 10B). These findings suggest that this set of galactose transporters is affected neither by the genetic background (WT or Y4588) nor by the carbon source.

2.7 Galactose is a Good Substrate for Industrial Processes Using Y. lipolytica

To confirm that galactose could be effectively used in industrial applications (e.g., the Y. lipolytica Y4588 strain could efficiently produce citric acid and lipids), batch cultures were raised in flasks and bioreactors. All the biomass, citric acid, and lipid production results are summarized in Table 4.

TABLE 4
Biomass, citric acid, and lipid production. Y. lipolytica W29 and Y4588 strains were grown
for 96 h in YNB medium containing 6% glucose or 6% galactose in flasks and bioreactors.

| | | | | | | | | |
|---|---|---|---|---|---|---|---|---|
| | X | Y_{X/S} | CA | Y_{CA/S} | Y_{CA/X} | FA | Y_{FA/S} | Y_{FA/X} |
| Parameter | g · L⁻¹ | g · g⁻¹ | g · L⁻¹ | g · g⁻¹ | g · g⁻¹ | g · L⁻¹ | g · g⁻¹ | g · g⁻¹ |

Flasks

| | | | | | | | | | |
|---|---|---|---|---|---|---|---|---|---|
| Glu 6% (C/N 60) | W29 | 11.15 | 0.24 | 4.18 | 0.09 | 0.37 | 1.92 | 0.041 | 0.172 |
| Gal 6% (C/N 60) | Y4588 | 10.90 | 0.21 | 18.17 | 0.35 | 1.67 | 1.87 | 0.036 | 0.172 |
| Gal 6% (C/N 100) | | 11.27 | 0.19 | 12.58 | 0.22 | 1.12 | 1.98 | 0.034 | 0,176 |
| Bioreactors | | 8.45 | 0.17 | 16.96 | 0.35 | 2.01 | 1.79 | 0.037 | 0.212 |
| Gal 6% (C/N 100) | Y4588 | 19.4 | 0.34 | 29.2 | 0.51 | 1.51 | 3.22 | 0.056 | 0.166 |

Symbols: X—dry biomass; FA—fatty acids; CA—citric acid; Y_{X/S}—yield of biomass from substrate consumed; Y_{CA/S}—yield of citric acid from substrate consumed; Y_{CA/X}—yield of citric acid from dry biomass; Y_{FA/S}—yield of fatty acids from substrate consumed; and Y_{FA/X}—yield of fatty acids from dry biomass. In this analysis, SD did not exceed 5%.

The Y. lipolytica wild-type (W29) and quadruple mutant (Y4588) strains produced similar amounts of biomass (˜11 g·L⁻¹) when grown in both glucose and galactose media with C/N ratio of 60. Biomass yield ranged from 0.19 to 0.24 g·g⁻¹. When the C/N ratio of the galactose medium was increased to 100, biomass yield decreased slightly (11%).

Interestingly, in glucose, citric acid biosynthesis and yield were around four times higher for Y4588 than for W29. Furthermore, Y4588 was able to produce three times more citric acid from galactose (C/N 60) than did W29 grown in glucose. Finally, when the C/N ratio was increased to 100, biomass and citric acid yield from galactose increased by 55% and 37%, respectively.

Galactose could also be used to efficiently produce lipids. In glucose with a C/N ratio of 60, W29 and Y4588 produced similar amounts of total lipids. Y4588 was able to produce comparable amounts of total lipids in galactose- and glucose-only media (Table 4). Lipid yields for W29 and Y4588 ranged between 0.034 and 0.041 g·g⁻¹. Additionally, increasing the C/N ratio to 100 improved lipid accumulation by 20%; it reached 0.212 g·g⁻¹. W29 had similar or lower yields when grown in glucose or fructose media with the same C/N ratio (Lazar et al., 2014).

To study Y4588's ability to produce citric acid and lipids in large quantities, the production was scaled up by using 2-L bioreactors. Overall, biomass, citric acid, and lipid production were dramatically improved (Table 4). Up to 19.4 g·L⁻¹ of biomass was produced under these conditions, representing an increase of 100%. Citric acid production reached 29.2 g·L⁻¹ and thus the conversion of substrate to citric acid was improved by 31%. Additionally, lipid production increased from 1.79 to 3.22 g·L⁻¹, which signifies that 34% more galactose was converted into lipids. The construction of a Y. lipolytica strain that is able to produce citric acid and lipids from galactose is a very important step in increasing the range of carbohydrate substrates used by this yeast for industrial applications.

### REFERENCES

André, A., et al., 2009. Eng. in Life Sci. 9:468-478.
Barth G. and Gaillardin C., 1996 Yarrowia lipolytica, In Nonconventional yeasts in biotechnology, vol. 1, K. Wolf, K. D. Breunig, and G. Barth (ed.), Springer-Verlag, Berlin, Germany, 313-388
Beopoulos A., et al., 2008. Applied Environmental Microbiology. 74: 7779-7789.
Beopoulos A., et al., 2012. Applied Microbiology and Biotechnology. 93: 1523-1537.
Blazeck, J., 2011. Appl. Environ Microbiol. 77: 7905-7914.
Blazeck, J., 2013. Appl. Microbiol Biotechnol. 97:3037-3052.
Blazeck J, et al., 2014. Nature Communications. 5: 3131, DOI: 10.1038/ncomms4131.
Bouffard G. G., et al., 1994. J Mol Biol. 162: 156-159.
Browse, J., et al., 1986, Anal. Biochem. 152:141-145.
Christensen U., et al., 2011. Appl Environ Microbiol. 77: 7084-7087.
Coelho M. A. Z., et al., 2010. Yarrowia lipolytica: an industrial workhorse. In: Méndez-Villas A (ed). Current Research, Technology and Education Topics in Applied Microbiology and Microbial Biotechnology. Vol. 2. Formatex, Badajoz, p 930.
Dear S., Staden R. 1991. Nucleic Acids Res. 19: 3907-3911.
Dulermo T., et al., 2013. Biochimica and Biophysica Acta. 1831: 1486-1495.
Fakas, S., et al., 2006, Appl Microbiol Biotechnol., 73:676-683.
Fickers P., et al., 2003. Journal of Microbiological Methods. 55: 727-737.
Frey P. A., 1996. The FASEB Journal. 10: 461-470.
Gaillardin C., Ribet A M. 1987. Current Genetics. 11: 369-375.
Gruben B. S., et al., 2012. FEBS Letters. 586: 3980-3985.
Guo, X., et al., 2000, J. Appl. Microbiol. 89:107-115.
Gysler, C., et al., 1990, Biotechn Techn. 4:285-290.
Hartl L., et al., 2007. The Journal of Biological Chemistry. 282: 18654-18659.
Hedfalk, K. 2012 ‘Codon Optimisation for Heterologous Gene Expression in Yeast’. In Springer Protocols: Methods in Molecular Biology. Recombinant protein production in yeast: methods and protocols. Volume 866 pp. 47-55. Springer Eds.
Holden H. M., et al., 2003. The Journal of Biological Chemistry. 278: 43885-43888.
Ito, H., et al., 1983, J Bacteriol. 153:163-168.
Klebe, R. J., et al., 1983, Gene. 25:333-341.
Koivistoinen O. M., et al., 2012. FEBS Letters. 586: 378-383.
Lazar Z., et al., 2014. Metabolic Engineering. 26: 89-99.
Le Dall, M. T., et al., 1994, Curr Genet. 26:38-44.
Liu X., et al., 2014. Appl Biochem Biotechnol. DOI 10.1007/s12010-014-1430-0
Madzak C., et al., 2000, J Mol Microbiol Biotechnol. 2:207-216.
Madzak, C., et al., 2004, J Biotechnol. 109:63-81.
Majumdar S., et al., Eur. J. Biochem. 2004. 271: 753-759.
Mauersberger S., et al., 2001. Journal of Bacteriology. 183: 5102-5109.
Meyer J., et al., 1991. Mol Cell Biol. 11:5454-5461.
Michely S., et al., 2013. PLOS one. 8: e63356 (1-10).
Mirończuk A. M., et al., 2014. Journal of Industrial Microbiology and Biotechnology. 41: 57-64.
Mojzita D., et al., 2012. J Biol Chem. 287: 26010-26018.
Müller S., et al., 1998. Yeast. 14:1267-1283.
Mumberg D., et al., 1995. Gene. 156: 119-122.
Orr-Weaver, T. L., et al., 1981, Proc. Natl. Acad. Sci. USA, 78:6354-6358.
Papanikolaou, S., et al., 2001, Antonie van Leeuwenhoek, 80:215-224.
Papanikolaou, S., et al., 2002, J. Appl. Microbiol. 92:737-744.
Papanikolaou, S., et al., 2008, Bioresour. Technol. 99:2419-2428.
Querol A., et al., 1992. Applied and Environmental Microbiology, 58: 2948-2953.
Sambrook J., Russell D W. 2001. Molecular Cloning: a Laboratory Manual (3rd ed.) Cold Spring Harbor Laboratory Press, New York
Schädel C., et al., 2010. Plant Physiology and Biochemistry. 48: 1-8.
Schmittgen T. D., Livak K. J., 2008. Nature Protocols. 3: 1101-1108.
Segawa T. and Fukasawa T., 1979. J. Biol. Chem. 254: 10707-10709.
Seiboth B., et al., 2002. Gene. 295: 143-149.
Sellick C. A., et al., 2008. Int Rev Cell Mol Biol. 269: 111-50.
Schell M. A. and Wilson D. B., 1977. J. Biol. Chem. 252: 1162-1166.
Slot J. C., Rokas A., 2010. PNAS. 107: 10136-10141.
Tai M., Stephanopoulos G. 2013. Metabolic engineering. 15: 1-9.
Tatusova, T. A. and Madden, T. L., 1999. FEMS Microbiol. Lett. 174:247-250.
van der Walt J. P., von Arx J A. 1980. Antonie van Leeuwenhoek. 46: 517-521.
Wang W., et al., 2014. PLOS One, DOI: 10.1371/journal.pone.0111443
Wong T. Y., Yao X. T., 1994. Applied and environmental Microbiology, 60: 2065-2068.
Xuan J. W., et al., 1990. Molecular and Cellular Biology. 10: 4795-4806.
Young E., et al., 2011. Appl Environ Microbiol. 77: 3311-3319.

1. A method for obtaining a Yarrowia strain capable of growing on D-galactose as sole carbon source, wherein said method comprises overexpressing in said strain a galactokinase (E.C 2.7.1.6) having at least 75% identity with the polypeptide of sequence SEQ ID NO: 2, a galactose-1-phosphate uridyl transferase (E.C 2.7.7.12) having at least 65% identity with the polypeptide of sequence SEQ ID NO: 4, an UDP-glucose-4 epimerase (E.C5.1.3.2) having at least 85% identity with the polypeptide of sequence SEQ ID NO: 6 and a galactose mutarotase (E.C5.1.3.3) having at least 45% identity with the polypeptide of sequence SEQ ID NO: 8.

2. The method according to claim 1, wherein 1, 2, 3 or 4 of said enzymes galactokinase, galactose-1-phosphate uridyl transferase, UDP-glucose-4 epimerase and galactose mutarotase, are from a Yarrowia strain.

3. The method according to claim 1, wherein said galactokinase has the consensus amino acid sequence SEQ ID NO: 9.

4. The method according to claim 1, wherein said galactokinase is selected from the group consisting of SEQ ID NO: 2, 10, 11, 12 and 13.

5. The method according to claim 1, wherein said galactose-1-phosphate uridyl transferase has the consensus amino acid sequence SEQ ID NO: 14.

6. The method according to claim 1, wherein said galactose-1-phosphate uridyl transferase is selected from the group consisting of SEQ ID NO: 4, 15, 16, 17 and 18.

7. The method according to claim 1, wherein said UDP-glucose-4 epimerase has the consensus amino acid sequence SEQ ID NO: 19.

8. The method according to claim 1, wherein said UDP-glucose-4 epimerase is selected from the group consisting of SEQ ID NO: 6, 20, 21, 22 and 23.

9. The method according to claim 1, wherein the amino acid sequence of said galactose mutarotase comprises, from the N-terminus to the C-terminus, the polypeptide fragments of SEQ ID NO: 24, 25 and 26.

10. The method according to claim 1, wherein said galactose mutarotase is selected from the group consisting of SEQ ID NO: 8, 27, 28 and 29.

11. The method according to claim 1, wherein said Yarrowia strain capable of growing on D-galactose as sole carbon source is a Y. lipolytica strain.

12. The method of claim 1, wherein it comprises transforming a Yarrowia cell with 1, 2, 3 or 4 recombinant DNA constructs for expressing the 4 enzymes as defined in claim 1.

13. A mutant Yarrowia strain, wherein a galactokinase having at least 75% identity with the polypeptide of sequence SEQ ID NO: 2, a galactose-1-phosphate uridyl transferase having at least 65% identity with the polypeptide of sequence SEQ ID NO: 4, an UDP-glucose-4 epimerase having at least 85% identity with the polypeptide of sequence SEQ ID NO: 6 and a galactose mutarotase having at least 45% identity with the polypeptide of sequence SEQ ID NO: 8 are overexpressed and wherein it is obtainable by the method of claim 1.

14. (canceled)

15. A method of producing lipids or citric acid, comprising a step of growing a mutant oleaginous yeast strain as defined in claim 13 on a medium comprising D-galactose.

16. An isolated mutant enzyme selected from the group consisting of:
a galactokinase (E.C 2.7.1.6) of SEQ ID NO: 10, 11, 12 or 13,
a galactose-1-phosphate uridyl transferase (E.C 2.7.7.12) of SEQ ID NO: 15, 16 or 17,
an UDP-glucose-4 epimerase (E.C 5.1.3.2) of SEQ ID NO: 20, 21, 22 or 23, and
a galactose mutarotase (E.C 5.1.3.3) of SEQ ID NO: 27, 28 or 29.


SEQUENCE LISTING (59 sequences)

SEQ ID NO: 1 - DNA, 1431 bp, Yarrowia lipolytica
  CDS (1)..(1431)
atgtctatcagcaccctgccgctggcgaacatctacgcggagccgaccaagcagcagccgcgctatgatcagctgactaagcggttttcggagctgtacgcagtctccagcccgactttggtgagatcgccgggccgagtcaacctcattggcgagcacattgactactcccacttcagtgtcatgcccatggcgattgaggatgacgtcatcatggccttcagcgttggcggcgactccattgagattgccaacatggaagacgacaagttcccggcagaaaagtacaacgttccgaccgccgaccaggtgctcactatcgaccagacaaaaacagtgtggaccaactacttcaaatgtggacacacagtgggccgaaagcatctgagagactctggagaggaggtaaaacccgtgggaatcaaggtgttggtccatggaactgtccccgacgcctgtggactgtcctcttccgctgcctttgtggtgtgctccactctcgtcactcttctgtcccagggcatgcaggaggtgtcacgtgagctgctcaccaggctctccatcacgtgcgagcagcttattggtgtcaacaccggcggtatggaccaggctgcctccatctacggccaactcaatcatactctcctggtggattttgctccaaccctcaaggccactccattccagttcccctcaacgacccccaagaccgtttttctggttgccaacacgctggtcaagtccaacaaacacgaaaccgcgcctcgaaactacaatctgcgggttgtggagatcactctggccgtcaacgtgcttctgaagagtctcaagggagtcaccattcctcagaacggaaacctcaacaagggaactcttcacggacttatgaaggctgttgatactgacctgcacggacttttggacctggtggaaactctgcccaaggagcctgtgactctgcaagaggtggccggcctgctgaacgtctctgaggacgacgtcaagaagcagtacttcattttccccgtcgaattcgacactctggctctctactcgcgggccaagcatgttgtcacagaggccattcgagtcaaggagtttcaaaacctgctgtccaagggcgagtctgaccccaggcagctcggcaagctcatgaacgagagtcaaaagtcgtgcgaccagctcttcaactgctcctgtcctgccatcgacaaggtgtgcgacattgtgctcaattctggagggtttggatcgcgtctgactggtgcggggtggggaggatctactgtccatctggttgaggaagccaagctgaacaacgtgattgacgctctgaagaaggaatactacagcgacatggatggtgtcgattacgatagcgccatggtcgtcactgtgcctggcgagggatgtgctattgttgatttgtaa

SEQ ID NO: 2 - protein, 476 aa, Yarrowia lipolytica
MSISTLPLANIYAEPTKQQPRYDQLTKRFSELYAVSSPTLVRSPGRVNLIGEHIDYSHFSVMPMAIEDDVIMAFSVGGDSIEIANMEDDKFPAEKYNVPTADQVLTIDQTKTVWTNYFKCGHTVGRKHLRDSGEEVKPVGIKVLVHGTVPDACGLSSSAAFVVCSTLVTLLSQGMQEVSRELLTRLSITCEQLIGVNTGGMDQAASIYGQLNHTLLVDFAPTLKATPFQFPSTTPKTVFLVANTLVKSNKHETAPRNYNLRVVEITLAVNVLLKSLKGVTIPQNGNLNKGTLHGLMKAVDTDLHGLLDLVETLPKEPVTLQEVAGLLNVSEDDVKKQYFIFPVEFDTLALYSRAKHVVTEAIRVKEFQNLLSKGESDPRQLGKLMNESQKSCDQLFNCSCPAIDKVCDIVLNSGGFGSRLTGAGWGGSTVHLVEEAKLNNVIDALKKEYYSDMDGVDYDSAMVVTVPGEGCAIVDL

SEQ ID NO: 3 - DNA, 1059 bp, Yarrowia lipolytica
  CDS (1)..(1059)
atgactcttgtggcatccgacattcctcacagacgatacaacccgctgcgacaatcatgggtgctggtgtcacctcatcgagctaagcgaccttggcagggacagcaagaagagcccaccaaggacacgaagcccgagtacgacgacaagtgctatctgtgtcctcgaaatgctcgaatcaacggggagatgaacccggactacaaggacactttcgtgtttgagaacgacttcagtgctgtttttgagaaaactgcggaagtggagaaggtcgttgaggaggcccaaaaggagcacacagagtctactgctctcgacctggaccttctgcgaaaagaagatgtgttcggatactgtcaggttatttgcttttcgcccaaacacaatgtgactcttgctacaatgactgttgagcagatcgaaaaggtgattgagacctggcagaaaatgtacacagaggctcagagtcgaggaatcaagtactcgctgatttttgaaaacaagggagctaccatgggctgctccaaccctcatgcccatggccaggtctggaacaccagtattgttcctgaagagccccagatggagtttgattcattgaagaaatacaaggacgaaaagaaccgtgatcttctggtcgattacgtcaacttggaaatgtccaaaaacgaacgaatcgtctaccagaatgcctcgtttctcgtggtggttccttactgggccgtctggccttttgagaccatgattgtgtccaaggaacgggtttcttccatcaaaaagctgaccaaggaacaagtccagcatcttgccgacgccatctcggtgatcacaaaaaagtacgacaagctcttcaacacctctttcccctactcaatgggtattcatcaatcatttgtggacgttgagaactgtccctacgagcatcttcggctcctgttctaccctcctcttctgcgatctgcctccgtcaagaagttccaggtcggattcgagatgctgggaatgccgcagcgagacattactgccgaaggggctgccaagcggttgtatgatttgtaa

SEQ ID NO: 4 - protein, 352 aa, Yarrowia lipolytica
MTLVASDIPHRRYNPLRQSWVLVSPHRAKRPWQGQQEEPTKDTKPEYDDKCYLCPRNARINGEMNPDYKDTFVFENDFSAVFEKTAEVEKVVEEAQKEHTESTALDLDLLRKEDVFGYCQVICFSPKHNVTLATMTVEQIEKVIETWQKMYTEAQSRGIKYSLIFENKGATMGCSNPHAHGQVWNTSIVPEEPQMEFDSLKKYKDEKNRDLLVDYVNLEMSKNERIVYQNASFLVVVPYWAVWPFETMIVSKERVSSIKKLTKEQVQHLADAISVITKKYDKLFNTSFPYSMGIHQSFVDVENCPYEHLRLLFYPPLLRSASVKKFQVGFEMLGMPQRDITAEGAAKRLYDL

SEQ ID NO: 5 - DNA, 1110 bp, Yarrowia lipolytica
  CDS (1)..(1110)
atgacccaggctgctgctgaacacgtgctcgtcaccggaggagccggatacatcggctctcacactgttattgctctggttgaggctggacgaaagccagtcattgtcgacaacctggacaactcgtgtgaggaggctgtgcgccgaatcgaacagattgtgggcgagaaaatccccttctttgaggttgatatcagagacactaacggtctggatgaagtgtacaagcagtacccaaccatcacctctgtgctgcatttcgctggtctcaaggccgtgggagaatctggccagattcctctggagtattacaactgcaacgtggcgggttccattaacctcctgcagtggtgccagaagcacaacatccacaactttgtcttctcgtcgtccgccaccgtctacggagacgtcacccggtttgagggcatgattcccattcctgaggagtgtcccaccggacccactaacccttacggacgaaccaagatgatgatcgaggacatcattcgagatcacgtgcgagccaatgctccctggaacgccgctattctgcgatacttcaatccgatgggtgcccatccctctggtctggttggagaggatcctcttggtatccccaacaacctgcttccttaccttgcccaggttgccgtgggccgacgagagaagctctttgtgtttggtgacgactacccctctcgagatggaactcccatccgagactacatccacgtgtgcgatcttgctgctggccacgtggctgcccttgagaagctcgaggctgtcgataaagagcgaaagcagctgccccaggggtcccccgagctcaagcacggtttctctcgagaatggaacctaggaaccggccacggatctactgtgcttgaggtcgtcaaggccttttccaacgccgtgggtaaggacctaccctacgaaatcaccggtcgacgagacggagacgttctggatctcaccgcgaaacccggccgagcaaactccgagctcaactggaaggcagagaagactctcgacgaaagctgctccgatctgtggagatggaccagcgagaacccctatggttacaaaaaggaccgtcatgacaagtaa

SEQ ID NO: 6 - protein, 369 aa, Yarrowia lipolytica
MTQAAAEHVLVTGGAGYIGSHTVIALVEAGRKPVIVDNLDNSCEEAVRRIEQIVGEKIPFFEVDIRDTNGLDEVYKQYPTITSVLHFAGLKAVGESGQIPLEYYNCNVAGSINLLQWCQKHNIHNFVFSSSATVYGDVTRFEGMIPIPEECPTGPTNPYGRTKMMIEDIIRDHVRANAPWNAAILRYFNPMGAHPSGLVGEDPLGIPNNLLPYLAQVAVGRREKLFVFGDDYPSRDGTPIRDYIHVCDLAAGHVAALEKLEAVDKERKQLPQGSPELKHGFSREWNLGTGHGSTVLEVVKAFSNAVGKDLPYEITGRRDGDVLDLTAKPGRANSELNWKAEKTLDESCSDLWRWTSENPYGYKKDRHDK

SEQ ID NO: 7 - DNA, 984 bp, Yarrowia lipolytica
  CDS (1)..(984)
atgaccttttgcaacacgggagcaacgctccagagctgggaaaatagcatcctcgggtttgaaaaccacacaaattacctgaaccgacaaaacccctactttggagctacaattggcccggtagctaaccggctataccaaccaaacggtatcctgttgcacggcggaaaagaaggctggtcctggaagaaatggaacaagagctcccccgccgctgctgaaaatagtaacgtgaccaaatttgagctcagagatacccacatcaccgcccacgtgacatatacgctgactaagtcgcccgacaagaagacgtcccaactggaaatcgaatactccgccaacagtaatgagctgggtaagagcatgccgattagcatgacaaatcacagctacttctgcttggacaaagacggtgccgaaggtgagggtcacgtgaccgtcgacgaccacgagctctggctgtgtaacgatcatgtgattgaacataaggacggtctgcaatcggagcctcctacgggcgagcttgtccgatgcgaggcattgttgaaggcgggggcgggtgagtcacgtgacacattctcgtcacttggtcgcttttcggacttgtcttcacttggtgggacgtccatctccctcaatggaactgttctcgaccacacttttgctgtcgataacgacaggctttcacgtgatgccaagctcgatacccgtgatgatccattagtcagcgttgcaattttgtcacgtggaaagacccgtctgaccgtcaagaccaccgagccctgtttccaggtctataccggcgactacaacaatgtggaactgcttcccggcgagtcacgtgactttggtcgacgttctggggttgcgattgagtgttctcgccccacgaatgcttggaatgttcctgggtggcagccgtgggttgatttgggtcagggggagtatggtgctaagattatctatgttttggagcgggagtga

SEQ ID NO: 8 - protein, 327 aa, Yarrowia lipolytica
MTFCNTGATLQSWENSILGFENHTNYLNRQNPYFGATIGPVANRLYQPNGILLHGGKEGWSWKKWNKSSPAAAENSNVTKFELRDTHITAHVTYTLTKSPDKKTSQLEIEYSANSNELGKSMPISMTNHSYFCLDKDGAEGEGHVTVDDHELWLCNDHVIEHKDGLQSEPPTGELVRCEALLKAGAGESRDTFSSLGRFSDLSSLGGTSISLNGTVLDHTFAVDNDRLSRDAKLDTRDDPLVSVAILSRGKTRLTVKTTEPCFQVYTGDYNNVELLPGESRDFGRRSGVAIECSRPTNAWNVPGWQPWVDLGQGEYGAKIIYVLERE

SEQ ID NO: 9 - protein, 483 aa, Artificial Sequence
  GAL1 consensus sequence
  VARIANT (2)..(2): Replace = Ser
  VARIANT (3)..(3): Replace = Ile
  VARIANT (4)..(4): Replace = Ser or Thr
  VARIANT (5)..(5): Replace = Thr
  VARIANT (7)..(7): Replace = Pro
  VARIANT (9)..(9): Replace = Ala or Ser
  VARIANT (10)..(10): Replace = Asn
  VARIANT (13)..(13): Replace = Ser
  VARIANT (14)..(14): Replace = Glu or Ser
  VARIANT (16)..(16): Replace = Thr, Ser or Glu
  VARIANT (17)..(17): Replace = Arg or Thr
  VARIANT (18)..(18): Replace = His or Ala
  VARIANT (19)..(19): Replace = Gln or Arg
  VARIANT (20)..(20): Replace = Pro, Thr or Lys
  VARIANT (23)..(23): Replace = Glu or Val
  VARIANT (24)..(24): Replace = Gln, Glu or Asn
  VARIANT (25)..(25): Replace = Leu
  VARIANT (26)..(26): Replace = Val
  VARIANT (27)..(27): Replace = Lys or Thr
  VARIANT (30)..(30): Replace = Ser, Thr or Arg
  VARIANT (31)..(31): Replace = Glu
  VARIANT (34)..(34): Replace = Pro or nothing
  VARIANT (35)..(35): Replace = Val
  VARIANT (36)..(36): Replace = Ser, Ala, Cys
  VARIANT (37)..(37): Replace = Ser or Lys
  VARIANT (38)..(38): Replace = Ala
  VARIANT (40)..(40): Replace = Leu or Val
  VARIANT (43)..(43): Replace = Ala
  VARIANT (67)..(67): Replace = Gly
  VARIANT (71)..(71): Replace = Ile
  VARIANT (75)..(75): Replace = Ser
  VARIANT (77)..(77): Replace = Gly or Glu
  VARIANT (80)..(80): Replace = Ser or Lys
  VARIANT (82)..(82): Replace = Glu or Arg
  VARIANT (83)..(83): Replace = Ile
  VARIANT (86)..(86): Replace = Met or Ile
  VARIANT (89)..(89): Replace = Glu
  VARIANT (93)..(93): Replace = Ser
  VARIANT (95)..(95): Replace = Lys or Ile
  VARIANT (97)..(97): Replace = Asn or His
  VARIANT (98)..(98): Replace = Val or Cys
  VARIANT (100)..(100): Replace = Thr, Ala or Asp
  VARIANT (101)..(101): Replace = Ala or Pro
  VARIANT (102)..(102): Replace = Asp, Thr or Asn
  VARIANT (103)..(103): Replace = Gln or Cys
  VARIANT (104)..(104): Replace = Glu
  VARIANT (106)..(106): Replace = Thr
  VARIANT (109)..(109): Replace = Gln
  VARIANT (110)..(110): Replace = Thr or Lys
  VARIANT (122)..(122): Replace = Phe
  VARIANT (125)..(125): Replace = Gly
  VARIANT (127)..(127): Replace = Lys
  VARIANT (128)..(128): Replace = Tyr
  VARIANT (129)..(129): Replace = Phe or Val
  VARIANT (130)..(130): Replace = Arg
  VARIANT (131)..(131): Replace = Asp or Glu
  VARIANT (132)..(132): Replace = Thr
  VARIANT (133)..(133): Replace = His
  VARIANT (134)..(134): Replace = Glu, Asp, or Gly
  VARIANT (135)..(135): Replace = Glu or Asp
  VARIANT (136)..(136): Replace = Leu or nothing
  VARIANT (137)..(137): Replace = Lys or Gln
  VARIANT (138)..(138): Replace = Pro
  VARIANT (139)..(139): Replace = Leu
  VARIANT (143)..(143): Replace = Ile
  VARIANT (169)..(169): Replace = Thr
  VARIANT (171)..(171): Replace = Leu or Met
  VARIANT (172)..(172): Replace = Ser
  VARIANT (173)..(173): Replace = Gln or Tyr
  VARIANT (175)..(175): Replace = nothing
  VARIANT (176)..(176): Replace = nothing
  VARIANT (177)..(177): Replace = nothing
  VARIANT (178)..(178): Replace = Met or Leu
  VARIANT (179)..(179): Replace = Gln, Lys or Thr
  VARIANT (180)..(180): Replace = Glu or Gln
  VARIANT (181)..(181): Replace = Val
  VARIANT (184)..(184): Replace = Glu
  VARIANT (188)..(188): Replace = Arg or Thr
  VARIANT (197)..(197): Replace = Val
  VARIANT (211)..(211): Replace = Tyr
  VARIANT (213)..(213): Replace = Val
  VARIANT (223)..(223): Replace = Ala
  VARIANT (225)..(225): Replace = Val or Lys
  VARIANT (227)..(227): Replace = Arg
  VARIANT (230)..(230): Replace = Pro
  VARIANT (232)..(232): Replace = Gln or Leu
  VARIANT (235)..(235): Replace = Ser or Ala
  VARIANT (237)..(237): Replace = Thr, Val or Ser
  VARIANT (239)..(239): Replace = Lys or Ser
  VARIANT (240)..(240): Replace = Ala
  VARIANT (249)..(249): Replace = Ile
  VARIANT (250)..(250): Replace = Arg
  VARIANT (270)..(270): Replace = Leu
  VARIANT (272)..(272): Replace = Val
  VARIANT (274)..(274): Replace = Ile
  VARIANT (275)..(275): Replace = Leu
  VARIANT (276)..(276): Replace = Leu
  VARIANT (277)..(277): Replace = His
  VARIANT (278)..(278): Replace = Ser or Gly
  VARIANT (280)..(280): Replace = Asn or nothing
  VARIANT (281)..(281): Replace = Asp
  VARIANT (282)..(282): Replace = Val
  VARIANT (283)..(283): Replace = Thr, Ser or Lys
  VARIANT (284)..(284): Replace = Ile
  VARIANT (285)..(285): Replace = Pro
  VARIANT (287)..(287): Replace = Asp
  VARIANT (295)..(295): Replace = Leu
  VARIANT (296)..(296): Replace = His or Arg
  VARIANT (299)..(299): Replace = Leu
  VARIANT (300)..(300): Replace = Lys or Glu
  VARIANT (302)..(302): Replace = Val or Cys
  VARIANT (303)..(303): Replace = Asp or Asn
  VARIANT (307)..(307): Replace =  Asn, His or Thr
  VARIANT (310)..(310): Replace =  Leu
  VARIANT (311)..(311): Replace =  Asp or Lys
  VARIANT (313)..(313): Replace =  Val
  VARIANT (314)..(314): Replace =  nothing
  VARIANT (315)..(315): Replace =  Phe
  VARIANT (316)..(316): Replace =  Thr
  VARIANT (319)..(319): Replace =  Lys
  VARIANT (320)..(320): Replace =  Asp
  VARIANT (321)..(321): Replace =  Pro
  VARIANT (322)..(322): Replace =  Leu
  VARIANT (323)..(323): Replace =  Thr
  VARIANT (325)..(325): Replace = Gln
  VARIANT (327)..(327): Replace = Val
  VARIANT (328)..(328): Replace = Ala
  VARIANT (329)..(329): Replace = Gly, Lys or Thr
  VARIANT (332)..(332): Replace = Asn, Asp or Glu
  VARIANT (333)..(333): Replace = Val or Ile
  VARIANT (335)..(335): Replace = Glu
  VARIANT (336)..(336): Replace = Asp or Glu
  VARIANT (337)..(337): Replace = Asp
  VARIANT (338)..(338): Replace = Ile
  VARIANT (339)..(339): Replace = Arg
  VARIANT (340)..(340): Replace = Lys
  VARIANT (341)..(341): Replace = Gln or Glu
  VARIANT (344)..(344): Replace = Ile
  VARIANT (346)..(346): Replace = Pro
  VARIANT (350)..(350): Replace = Lys
  VARIANT (351)..(351): Replace = Thr or Ser
  VARIANT (353)..(353): Replace = Ala
  VARIANT (356)..(356): Replace = Ser or Ala
  VARIANT (363)..(363): Replace = Ser
  VARIANT (366)..(366): Replace = Leu
  VARIANT (369)..(369): Replace = Lys or Met
  VARIANT (370)..(370): Replace = Lys
  VARIANT (372)..(372): Replace = Arg
  VARIANT (373)..(373): Replace = Asn or Lys
  VARIANT (374)..(374): Replace = Leu or Lys
  VARIANT (377)..(377): Replace = Lys, Leu or Glu
  VARIANT (379)..(379): Replace = Glu or Val
  VARIANT (381)..(381): Replace = Asn
  VARIANT (383)..(383): Replace = Arg, Lys or Ile
  VARIANT (384)..(384): Replace = Gln or Glu
  VARIANT (387)..(387): Replace = Lys
  VARIANT (391)..(391): Replace = Glu
  VARIANT (394)..(394): Replace = Lys or Glu
  VARIANT (397)..(397): Replace = Asp
  VARIANT (398)..(398): Replace = Gln or Glu
  VARIANT (401)..(401): Replace = Asn or Asp
  VARIANT (405)..(405): Replace = Pro
  VARIANT (406)..(406): Replace = Glu
  VARIANT (408)..(408): Replace = Glu
  VARIANT (409)..(409): Replace = Lys or Glu
  VARIANT (412)..(412): Replace = Asp or Glu
  VARIANT (416)..(416): Replace = Asn or Lys
  VARIANT (417)..(417): Replace = Ala
  VARIANT (420)..(420): Replace = Phe
  VARIANT (438)..(438): Replace = Gln
  VARIANT (439)..(439): Replace = Glu or Arg
  VARIANT (440)..(440): Replace = Ala or Asp
  VARIANT (442)..(442): Replace = Val
  VARIANT (443)..(443): Replace = Asn, Gln or Asp
  VARIANT (444)..(444): Replace = Asn or Tyr
  VARIANT (447)..(447): Replace = Asp or Gly
  VARIANT (448)..(448): Replace = Ala or Thr
  VARIANT (451)..(451): Replace = Ser
  VARIANT (455)..(455): Replace = Glu
  VARIANT (456)..(456): Replace = Asp or Arg
  VARIANT (457)..(457): Replace = Met or Leu
  VARIANT (458)..(458): Replace = Asp, Glu or Asn
  VARIANT (459)..(459): Replace = Gly or Lys
  VARIANT (460)..(460): Replace = nothing
  VARIANT (461)..(461): Replace = Lys
  VARIANT (462)..(462): Replace = Tyr or Ala
  VARIANT (464)..(464): Replace = Ser or Asn
  VARIANT (466)..(466): Replace = Ile
  VARIANT (467)..(467): Replace = Leu
  VARIANT (473)..(473): Replace = Gln
  VARIANT (476)..(476): Replace = Cys
  VARIANT (478)..(478): Replace = Ile or Val
  VARIANT (480)..(480): Replace = Leu, Cys or Phe
  VARIANT (481)..(481): Replace = Lys, Ser or nothing
  VARIANT (482)..(482): Replace = nothing
  VARIANT (483)..(483): Replace = nothing
MNVAKLGLEKIYADPAKQSARYDDITARFKQLYAGQTPTIVRSPGRVNLIGEHIDYSHFSVMPMAIEDDVVMAFCVAGDTIDVANLEDDKFPAESYDIPSGEEVLEIDTQKTVWTNYFKCGHTVARRHLNLSGKSVRDVGIKVLVHGTVPDACGLSSSAAFVVCSTLVSLIACGGGAEDSISRDLLTKLSITCEQLIGVNTGGMDQAASIFGQLNHTLLVDFSPTLKATSFKFPETRPETVFLVANTLVKSNKHETAPRNYNLRVVEITIAANVIAKKLKGLQLVQNGNLNKGTFNGLMQAAGTDLAGLEGLIDEHLPREGVSLEEASELLGASVAEVKESYFVFHVEFDKLQLYKRAKHVVTEAIRVLEFQSMLSSGTSDPSKLGNLMNLSQQSCRDLFECSCDAIDRVCKIVLASGGLGSRLTGAGWGGSTVHLVEKEKLSSVIESLKKEYYSGIGEVDFDTAMVVTVPGEGTAFVDIQLH

SEQ ID NO: 10 - protein, 476 aa, Yarrowia galli
MSISTLPLSKIYADPSKHQTRYEELTKRFSQLYPGSTATLVRSPGRVNLIGEHIDYSHFSVMPMAIEDDVIMAFSVGGDSIEIANMEDEKFPAEKYDIPTATEVLTIDQTKTVWTNYFKCGHTVGRKYLRESGEEVKPVGIKVLVHGTVPDACGLSSSAAFVVCSTLVTLLSQGMKEVSRDLLTKLSITCEQLIGVNTGGMDQAASIYGQLNHTLLVDFAPTLKATPFQFPATTPKTVFLVANTLVKSNKHETAPRNYNLRVVEITLAVNVLLKSLNGVSIPQNGNLNKGTLHGLMKAVGTDLNGLLDLVETLPKEPVTLQEVAKLLDISEEDVKKQYFIFPVEFDTLALYARAKHVVTEAIRVLEFQSLLSKGESDPKELGKLMNESQKSCDQLFNCSCPAIEKVCEIVLKSGGFGSRLTGAGWGGSTVHLVEEAKLNNVIGALKKEYYSDMEGVDYDSAMVVTVPGEGCAIVDL

SEQ ID NO: 11 - protein, 476 aa, Yarrowia yakushimensis
MSIATLPLSKIYADPARHQARYEQLTKRFTELYGAKPTVVRSPGRVNLIGEHIDYSHFSVMPMAIEDDVIMAFSVGGDSIEIANIEDEKFPAEKYDIPAPNEVLTIDQKKTVWTNYFKCGHTVARKHFRDSGDDVKPVGIKILVHGTVPDACGLSSSAAFVVCSTLVTLLSQGLTEVSRDLLTTLSITCEQLIGVNTGGMDQAASIYGQLNHTLLVDFAPVLKATPFLFPSTVPKTVFLVANTLVKSNKHETAPRNYNLRVVEITLAVNVLLKGLKDVTIPQNGNLNKGTLRGLMEAVGTDLHGLLDLVETLPKEPVTLQEVAKLLDISEEDVKKQYFIFPVEFDTLALYSRAKHVVTEALRVLEFQKKLSLGESDPKQLGKLMNESQKSCDQLFNCSCPAIEEVCEIVLKSGGLGSRLTGAGWGGSTVHLVEEAKLQNVIDALKKEYYSDMEGVDYDNAMVVTVPGEGCAVVDCK

SEQ ID NO: 12 - protein, 478 aa, Yarrowia alimentaria
MNVAKLGLEKIYADPAKQSARYDDITARFKQLYGQTPTIVRSPGRVNLIGEHIDYSHFSVMPMAIEDDVVMAFCVAGDTIDVANLEDDKFPAESYDIPSGEEVLEIDTQKTVWTNYFKCGHTVARRHLNLSGKSRDVGIKVLVHGTVPDACGLSSSAAFVVCSTLVSLIACGGGAEDSISRDLLTKLSITCEQLIGVNTGGMDQAASIFGQLNHTLLVDFSPTLKATSFKFPETRPETVFLVANTLVKSNKHETAPRNYNLRVVEITIAANVIAKKLGLQLVQNGNLNKGTFNGLMQAAGTDLAGLEGLIDEHLPREGVSLEEASELLGASVAEVKESYFVFHVEFDKLQLYKRAKHVVTEAIRVLEFQSMLSSGTSDPSKLGNLMNLSQQSCRDLFECSCDAIDRVCKIVLASGGLGSRLTGAGWGGSTVHLVEKEKLSSVIESLKKEYYSGIGEVDFDTAMVVTVPGEGTAFVDIQ

SEQ ID NO: 13 - protein, 477 aa, Yarrowia phangngensis
MSITKLPLEKIYSSPETARKRYVNIVTRFRELYGCTPTLVRAPGRVNLIGEHIDYSHFSVMPMAIGDDVIMAFCVEGDKIRIANLEDEKFPSEIYHCPDADCELEIDQTKTVWTNYFKCGFTVGRKYVRETHGSLQPLGIKILVHGTVPDACGLSSSAAFVVCSTLVTLMAYGETQVSRELLTKLSITCEQLVGVNTGGMDQAASIYGVLNHTLLVDFAPKLRATPFLFPETSPSAVFLVANTLIRSNKHETAPRNYNLRVVEITLAVNILAHKLGLKIPQDGNLNKGTFHGLLEACNTDLTGLLKLVDFHLPRDPLTLEEVSTLLEISEDEIRKEYFVFPVEFKSLALYSRAKHVVSEAIRVMKFRKKLSEGVSNPIELGKLMNESQESCRELFDCSCPEIDRVCEIVLKAGGFGSRLTGAGWGGSTVHLVQRDKVDYVIDTLKSEYYERLNKKADSAILVTVPGQGCAIVDFSLH

SEQ ID NO: 14 - protein, 355 aa, Artificial Sequence
  GAL7 consensus sequence
  VARIANT (4)..(4): Replace = Val or Glu
  VARIANT (5)..(5): Replace = Ala
  VARIANT (6)..(6): Replace = Ser or Phe
  VARIANT (18)..(18): Replace = Gln
  VARIANT (40)..(40): Replace = Thr or Phe
  VARIANT (43)..(43): Replace = Thr or Asp
  VARIANT (49)..(49): Replace = Glu
  VARIANT (56)..(56): Replace = Cys
  VARIANT (58)..(58): Replace = Ala or Glu
  VARIANT (60)..(60): Replace = Val
  VARIANT (64)..(64): Replace = Met or Lys
  VARIANT (69)..(69): Replace = Lys
  VARIANT (70)..(70): Replace = Asp
  VARIANT (72)..(72): Replace = Tyr
  VARIANT (75)..(75): Replace = Asn
  VARIANT (83)..(83): Replace = Glu
  VARIANT (84)..(84): Replace = Arg
  VARIANT (86)..(86): Replace = Ala or Asp
  VARIANT (88)..(88): Replace = Val
  VARIANT (90)..(90): Replace = Lys or Glu
  VARIANT (91)..(91): Replace = Val
  VARIANT (92)..(92): Replace = Val, Ile or Leu
  VARIANT (93)..(93): Replace = Glu or Arg
  VARIANT (95)..(95): Replace = nothing
  VARIANT (96)..(96): Replace = nothing
  VARIANT (97)..(97): Replace = Ala
  VARIANT (98)..(98): Replace = Gln, Ser or Asn
  VARIANT (99)..(99): Replace = Lys or Glu
  VARIANT (100)..(100): Replace = nothing
  VARIANT (101)..(101): Replace = Glu or Lys
  VARIANT (102)..(102): Replace = His, Glu, Gln or Asn
  VARIANT (103)..(103): Replace = Thr
  VARIANT (104)..(104): Replace = Glu, Lys or Gly
  VARIANT (105)..(105): Replace = Ser, Pro or Phe
  VARIANT (106)..(106): Replace = Thr, Ser or His
  VARIANT (107)..(107): Replace = Ala or Glu
  VARIANT (108)..(108): Replace = Leu
  VARIANT (109)..(109): Replace = Asp
  VARIANT (110)..(110): Replace = Leu, Arg or Ser
  VARIANT (111)..(111): Replace = Leu or Asn
  VARIANT (114)..(114): Replace = Gln or Asn
  VARIANT (115)..(115): Replace = Lys or Arg
  VARIANT (116)..(116): Replace = Glu
  VARIANT (117)..(117): Replace = Asp or Glu
  VARIANT (118)..(118): Replace = Val
  VARIANT (137)..(137): Replace = Ser
  VARIANT (140)..(140): Replace = Pro
  VARIANT (141)..(141): Replace = Glu or Thr
  VARIANT (144)..(144): Replace = Gln
  VARIANT (145)..(145): Replace = Lys, Arg or Ala
  VARIANT (147)..(147): Replace = Val
  VARIANT (148)..(148): Replace = Glu, Ala or Asn
  VARIANT (151)..(151): Replace = Arg
  VARIANT (152)..(152): Replace = Lys
  VARIANT (153)..(153): Replace = Ile
  VARIANT (155)..(155): Replace = Thr or Glu
  VARIANT (159)..(159): Replace = Ser or Arg
  VARIANT (160)..(160): Replace = Lys
  VARIANT (161)..(161): Replace = Glu
  VARIANT (173)..(173): Replace = Ser
  VARIANT (174)..(174): Replace = Ala
  VARIANT (184)..(184): Replace = Cys
  VARIANT (191)..(191): Replace = Ile
  VARIANT (194)..(194): Replace = Asp
  VARIANT (198)..(198): Replace = Leu
  VARIANT (200)..(200): Replace = Phe
  VARIANT (201)..(201): Replace = Asp
  VARIANT (202)..(202): Replace = Ser
  VARIANT (204)..(204): Replace = Lys
  VARIANT (205)..(205): Replace = Lys
  VARIANT (208)..(208): Replace = Glu
  VARIANT (209)..(209): Replace = Glu
  VARIANT (210)..(210): Replace = Asn
  VARIANT (211)..(211): Replace = Asn or Lys
  VARIANT (219)..(219): Replace = Val
  VARIANT (220)..(220): Replace = Asn, His, Gln or Glu
  VARIANT (223)..(223): Replace = Met
  VARIANT (224)..(224): Replace = Ser or Gln
  VARIANT (226)..(226): Replace = Asn or Glu
  VARIANT (229)..(229): Replace = Ile
  VARIANT (232)..(232): Replace = Gln
  VARIANT (234)..(234): Replace = Ala or Glu
  VARIANT (253)..(253): Replace = Val
  VARIANT (256)..(256): Replace = Glu
  VARIANT (258)..(258): Replace = Val
  VARIANT (259)..(259): Replace = Ser
  VARIANT (261)..(261): Replace = Ile
  VARIANT (262)..(262): Replace = Lys
  VARIANT (265)..(265): Replace = Ser
  VARIANT (266)..(266): Replace = Gln or Asn
  VARIANT (267)..(267): Replace = His or Glu
  VARIANT (269)..(269): Replace = Ala
  VARIANT (270)..(270): Replace = Gln, Glu or Ser
  VARIANT (271)..(271): Replace = His
  VARIANT (275)..(275): Replace = Ser
  VARIANT (279)..(279): Replace = Ile
  VARIANT (281)..(281): Replace = Lys
  VARIANT (285)..(285): Replace = Asp
  VARIANT (288)..(288): Replace = Lys
  VARIANT (304)..(304): Replace = Val
  VARIANT (305)..(305): Replace = Glu
  VARIANT (306)..(306): Replace = Asp
  VARIANT (308)..(308): Replace = Pro
  VARIANT (345)..(345): Replace = Thr
  VARIANT (347)..(347): Replace = Glu
  VARIANT (350)..(350): Replace = Lys
  VARIANT (353)..(353): Replace = Tyr, Phe or Arg
  VARIANT (354)..(354): Replace = Asp or Glu
MTLSPADIPHRRYNPLRKSWVLVSPHRAKRPWQGQQEEPVKDSKPEYDDKCYLCPRNKRINGELNPDYQSTFVFENDFSAVFPKTEEIESLMKEEKSEQKTSSSLAGIEADLLRQDPTFGYCQVICFSPKHNVTLATMTVPQIEQVIYTWQDMYREAQTRQIKYSLIFENKGATMGCSNPHAHGQVWNTSVVPEEPQMEYNALREYKDSKSRDLLVDYAALELEKKERVVYENSSFLVVVPYWAVWPFETMIFSKQRIASVIKLTKPQVADLADAISVLTRKYDKLFNTSFPYSMGIHQSFVDEPNCEYEHLRLLFYPPLLRSASVKKFQVGFEMLGMPQRDITAEGAAQRLKGL

SEQ ID NO: 15 - protein, 353 aa, Yarrowia galli
MTLVASDIPHRRYNPLRQSWVLVSPHRAKRPWQGQQEEPTKDTKPEYDDKCYLCPRNARINGEMNPDYKDTFVFENDFSAVFEKTAEVEKVIEEAQKKEESKPSELELDLLRKDDVFGYCQVICFSPKHNVTLATMTVEQIQRVIETWQKMYTEAQSRGIKYSLIFENKGATMGCSNPHAHGQVWNTSVVPEEPQMEFDSLKKYKDEKKRDLLVDYVHLEMSKNERIVYQNASFLVVVPYWAVWPFETMIVSKERVSSIKKLTQEQVEHLADAISVLTKKYDKLFNTSFPYSMGIHQSFVDVEDCPYEHLRLLFYPPLLRSASVKKFQVGFEMLGMPQRDITAEGAAKRLFDL

SEQ ID NO: 16 - protein, 352 aa, Yarrowia yakushimensis
MTLVASDIPHRRYNPLRQSWVLVSPHRAKRPWQGQQEEPTKDTKPEYDEKCYLCPRNARINGEMNPDYKDTFVFENDFSAVFEKTAEVEKVIEEASKKQSESSELERLLLQKEDVFGYCQVICFSPKHNVTLATMTVEQIEKVIATWQKMYTEAQSRGIKYSLIFENKGATMGCSNPHAHGQVWNTSVVPEEPQMEYDSLKKYKEENKRDLLVDYVQLELSKNERIVYQNASFLVVVPYWAVWPFETMIVSKERVSSIKKLTKEQAEHLADAISVLTKKYDKLFNTSFPYSMGIHQSFVDVEDCPYEHLRLLFYPPLLRSASVKKFQVGFEMLGMPQRDITAEGAAKRLYEL

SEQ ID NO: 17 - protein, 352 aa, Yarrowia phangngensis
MTLEAFDIPHRRYNPLRQSWVLVSPHRAKRPWQGQQEEPFKDDKPEYDEKCYLCPCNERVNGEKNPDYQDTYVFNNDFSAVFERTDEVEELLRESNETNSGFHEIESNLLNREEVFGYCQVICFSPKHNVTLASMTPTQIEAVVNTWRKIYEEAQRKGIKYSLIFENKGSAMGCSNPHAHCQVWNTSVVPDEPQLEFNSLKKYKDEKKRDLLVDYVGLELQKEERIVYQNESFLVVVPYWAVWPFETMIVSKERISSIKKLSNHQVSDLADSISVLTRKYDDLFKTSFPYSMGIHQSFVDVEDCPYEHLRLLFYPPLLRSASVKKFQVGFEMLGMPQRDITTEEAAKRLRDL

SEQ ID NO: 18 - protein, 355 aa, Yarrowia alimentaria; identical to SEQ ID NO: 14

SEQ ID NO: 19 - protein, 369 aa, Artificial Sequence; identical to SEQ ID NO: 6
  GAL10E consensus sequence
  VARIANT (4)..(4): Replace = Thr
  VARIANT (5)..(5): Replace = Thr or nothing
  VARIANT (6)..(6): Replace = nothing
  VARIANT (8)..(8): Replace = Tyr
  VARIANT (25)..(25): Replace = Ser
  VARIANT (27)..(27): Replace = Ile
  VARIANT (34)..(34): Replace = Ile
  VARIANT (44)..(44): Replace = Ile or Val
  VARIANT (45)..(45): Replace = Asp or Gln
  VARIANT (48)..(48): Replace = Ser or Gln
  VARIANT (52)..(52): Replace = His or Glu
  VARIANT (57)..(57): Replace = Thr
  VARIANT (58)..(58): Replace = Val
  VARIANT (62)..(62): Replace = Lys
  VARIANT (65)..(65): Replace = Val
  VARIANT (69)..(69): Replace = Lys
  VARIANT (73)..(73): Replace = Ala
  VARIANT (77)..(77): Replace = Glu
  VARIANT (78)..(78): Replace = Phe
  VARIANT (98)..(98): Replace = Glu
  VARIANT (99)..(99): Replace = Lys
  VARIANT (105)..(105): Replace = Phe
  VARIANT (109)..(109): Replace = Thr
  VARIANT (111)..(111): Replace = Thr
  VARIANT (119)..(119): Replace = His
  VARIANT (120)..(120): Replace = Ser
  VARIANT (122)..(122): Replace = Asp
  VARIANT (154)..(154): Replace = Asp
  VARIANT (166)..(166): Replace = Val
  VARIANT (169)..(169): Replace = Val
  VARIANT (178)..(178): Replace = Val or Pro
  VARIANT (179)..(179): Replace = Ala or Ser
  VARIANT (184)..(184): Replace = Leu
  VARIANT (213)..(213): Replace = Phe
  VARIANT (230)..(230): Replace = Asn
  VARIANT (235)..(235): Replace = Lys
  VARIANT (258)..(258): Replace = Asn, Asp or Lys
  VARIANT (262)..(262): Replace = Ser
  VARIANT (264)..(264): Replace = Glu or Gln
  VARIANT (265)..(265): Replace = His
  VARIANT (266)..(266): Replace = Gln
  VARIANT (268)..(268): Replace = Gln
  VARIANT (269)..(269): Replace = Glu
  VARIANT (272)..(272): Replace = Glu or Lys
  VARIANT (273)..(273): Replace = Asp
  VARIANT (275)..(275): Replace = Ala
  VARIANT (291)..(291): Replace = Leu
  VARIANT (296)..(296): Replace = Met
  VARIANT (299)..(299): Replace = Ile
  VARIANT (300)..(300): Replace = Gln or Asp
  VARIANT (303)..(303): Replace = Cys
  VARIANT (304)..(304): Replace = Lys
  VARIANT (309)..(309): Replace = Thr
  VARIANT (314)..(314): Replace = Val
  VARIANT (315)..(315): Replace = Ala
  VARIANT (330)..(330): Replace = Asp
  VARIANT (334)..(334): Replace = Thr
  VARIANT (337)..(337): Replace = Glu, Ser or Gln
  VARIANT (339)..(339): Replace = Arg
  VARIANT (341)..(341): Replace = Lys
  VARIANT (342)..(342): Replace = Gln
  VARIANT (344)..(344): Replace = Met
  VARIANT (346)..(346): Replace = Lys
  VARIANT (349)..(349): Replace = Ala, Thr or Arg
  VARIANT (353)..(353): Replace = Lys
  VARIANT (357)..(357): Replace = Gln
  VARIANT (364)..(364): Replace = Glu
  VARIANT (365)..(365): Replace = Glu
  VARIANT (366)..(366): Replace = Glu
  VARIANT (367)..(367): Replace = Lys
  VARIANT (368)..(368): Replace = nothing
  VARIANT (369)..(369): Replace = Glu or nothing

SEQ ID NO: 20 - protein, 369 aa, Yarrowia galli
MTQAAAEHVLVTGGAGYIGSHTVIALVEAGRKPVIVDNLDNSCEEAVRRIEQIVGEKIPFFEVDIRDTNGLDEVYKQYPTITSVLHFAGLKAVGESGQIPLEYYFCNVAGSINLLQWCQKHDIHNFVFSSSATVYGDVTRFEGMIPIPEECPTGPTNPYGRTKMMIEDIIRDHVRANAPWNAAILRYFNPMGAHPSGLVGEDPLGIPNNLLPYLAQVAVGRREKLFVFGDDYPSRDGTPIRDYIHVCDLAAGHVAALNKLEAVDKERKQLPEDSAELKHGFSREWNLGTGHGSTVLEVVKAFSKAVGKDLPYEIAGRRDGDVLDLTAKPGRANSELEWKAEKTLDESCSDLWRWTSENPYGYKKDRHDK

SEQ ID NO: 21 - protein, 368 aa, Yarrowia yakushimensis
MTQATEYVLVTGGAGYIGSHTVIALVEAGRKPIIVDNLDNSCEDAVSRIEHIVGETIPFFKVDIRDTNGLDEVYKQFPTITSVLHFAGLKAVGESGQIPLEYYNCNVTGSINLLQWCQKHNIHNFVFSSSATVYGDVTRFEGMIPIPEECPTGPTNPYGRTKMMVEDIIRDHVRANVPWNAAILRYFNPMGAHPSGLVGEDPLGIPNNLLPYLAQVAVGRREKLFVFGNDYPSRDGTPIRDYIHVCDLAAGHVAALDKLEAVDKERKQLPEGSPELKHGFSREWNLGTGHGSTVLEVVQAFSKAVGKDLPYEITGRRDGDVLDLTAKPGRANTELNWRAKQTLDKSCADLWRWTSENPYGYKKDRHDE

SEQ ID NO: 22 - protein, 365 aa, Yarrowia phangngensis
MTQTEHVLVTGGAGYIGSHTVISLVEAGRKPIIVDNLDNSCIQAVQRIEEIVGEKIPFFEVDVRDTKGLDEVYKQYPTITSVLHFAGLKAVGESGEIPLEYYFCNVAGSINLLQWCHKHNIHNFVFSSSATVYGDVTRFEGMIPIPEECPTGPTNPYGRTKMMIEDVIRDHVRANPAWNAAILRYFNPMGAHPSGLVGEDPLGIPNNLLPYLAQVAVGRREKLFVFGNDYPSRDGTPIRDYIHVCDLAAGHVAALNKLEAVEKQRKELPKDSAELKHGFSREWNLGTGHGSTVLEVVDAFCKAVGKTLPYEITGRRDGDVLDLTAKPGRANSELSWKAEKTLDESCTDLWRWTSQNPYGYKKEEK

SEQ ID NO: 23 - protein, 366 aa, Yarrowia alimentaria
MTQTEHVLVTGGAGYIGSHTVIALIEAGRKPVIVDNLDNSCVEAVRRIEQIVGEKVPFFEVDIRDTNGLDAVYKEFPTITSVLHFAGLKAVGESGQKPLEYYFCNVAGTINLLQWCHSHNIHNFVFSSSATVYGDVTRFEGMIPIPEECPTDPTNPYGRTKMMIEDIIRDHVRANPSWNAALLRYFNPMGAHPSGLVGEDPLGIPNNLLPFLAQVAVGRREKLFVFGNDYPSKDGTPIRDYIHVCDLAAGHVAALKKLESVQHERQELPKDSPELKHGFSREWNLGTGLGSTVMEVIKAFSKAVGKDLPYEVAGRRDGDVLDLTAKPDRANTELQWKAEKTMDESCRDLWKWTSENPYGYKEDRHD

SEQ ID NO: 24 - protein, 26 aa, Artificial Sequence
  Fragment of GAL10M
  VARIANT (3)..(3): Replace = Phe
  VARIANT (17)..(17): Replace = Asp or Asn
  VARIANT (19)..(19): Replace = Glu, Asp or Gln
  VARIANT (22)..(22): Replace = Val or Thr
NPYFGATIGPVANRLYQPNGILLHGG

SEQ ID NO: 25 - protein, 24 aa, Artificial Sequence
  Fragment of GAL10M
  VARIANT (3)..(3): Replace = Leu
  VARIANT (4)..(4): Replace = Asn
  VARIANT (6)..(6): Replace = Pro
  VARIANT (12)..(12): Replace = Glu
  VARIANT (17)..(17): Replace = Thr or Lys
  VARIANT (20)..(20): Replace = Asp or Val
  VARIANT (23)..(23): Replace = Lys
  VARIANT (24)..(24): Replace = Asp
RLSRDAKLDTRDDPLVSVAILSRG

SEQ ID NO: 26 - protein, 33 aa, Artificial Sequence
  Fragment of GAL10M
  VARIANT (2)..(2): Replace = Asn
  VARIANT (4)..(4): Replace = Ser
  VARIANT (7)..(7): Replace = Ala or Ser
  VARIANT (9)..(9): Replace = Ala
  VARIANT (15)..(15): Replace = Ala
  VARIANT (16)..(16): Replace = Ala
  VARIANT (23)..(23): Replace = His or Tyr
  VARIANT (26)..(26): Replace = Gln or Glu
  VARIANT (28)..(28): Replace = Lys or Arg
  VARIANT (29)..(29): Replace = Asn, Asp or Lys
  VARIANT (31)..(31): Replace = Thr
  VARIANT (33)..(33): Replace = Met
ESRDFGRRSGVAIECSRPTNAWNVPGWQPWVDL

SEQ ID NO: 27 - protein, 329 aa, Yarrowia galli
MTLCNTGATLQSWENSVLGFSNADNYVSSENPFFGATIGPVANRLYQPEGIVLHGGKESWAWKKWTRTEEEGHVTKFELRDTEITAHVTYTLTKPDSTTTQLEIEYSAKSMDGRSTPISMTNHSYFCLDQDSEGHVTVDDHELWLCNDHVIEHKDGLQSEAPTGELVCCEALERAELVKSRDQIESREIKQSRDISNDSTIPLPGSSRAFSLAGLSLDHTFAVNNQRLSRDPKLDTRDDPLVTVADLSRDKTRLIVKTTEPCFQVYTGDYNNVSQLPGESRDFGARSGVAIECSRPTNAWNVPQWKNWVDLGQGEYGAKIVYVLEKRGD

SEQ ID NO: 28 - protein, 319 aa, Yarrowia yakushimensis
MTFSATGATLQSWENCILGFENDANYLNNQNPYFGATIGPVANRLYDPDGIVLHGGEEGWSWKNWKKIEETEKVTKFELNDDRITAHVIYTLTNPHPNTSQLEIEYSALAKEKDANLPINMTNHSYFCLPPQSGHVTVDDHDLWLCNTQVLEHKDGLQSEPPTGKLIVCDPLKEVRSRDENNHDDVGSHDSNSPSHAVQMSLNGMALDHTFAVDNERLSRDAKLDTRDDPLVTVAVLSRDSKTRLTVKTTEPCFQVYTGDFISVDQLSGESRDFGSRAGVAIECSRPTNAWHVPEWRDWVDLSKGVYGSKIVYILEREE

SEQ ID NO: 29 - protein, 282 aa, Yarrowia alimentaria
MYSDIGAAVKALDGVVLGFSNDENYLKPENPFFGATIGPVANRLYNPQGITLHGGANSWAFKTWSRSENGNVTTFDLVDGDVRAQVKYTLVKTKTATTLEIEFWANSSGKSIPLSMANHCYFSLNGESTIDDSDLWLCSDKVIEHGNDSQNEAPTGQLKTFKRASNALTTPIDHTFVIDEQRLLNDAKLDTREDPLVKVAVLSKGNVSVTVETTEPVFQVYTGDYNNVPRFENENRSFGARSGVAIEAARPTNAWYVPQWKKWTDMAQGDYGSRTVYTVKRG

SEQ ID NO: 30 - DNA, 41 bp, Artificial Sequence
  Primer
gcgcggatccatgactaaatctcattcagaagaagtgattg

SEQ ID NO: 31 - DNA, 40 bp, Artificial Sequence
  Primer
gcgccctaggttataattcatatagacagctgcccaatgc

SEQ ID NO: 32 - DNA, 29 bp, Artificial Sequence
  Primer
gcgcggatccatgactgctgaagaatttg

SEQ ID NO: 33 - DNA, 35 bp, Artificial Sequence
  Primer
gcgccctaggttacagtctttgtagataatgaatc

SEQ ID NO: 34 - DNA, 30 bp, Artificial Sequence
  Primer
gcgcggatccatgacagctcagttacaaag

SEQ ID NO: 35 - DNA, 32 bp, Artificial Sequence
  Primer
gcgccctaggtcaggaaaatctgtagacaatc

SEQ ID NO: 36 - DNA, 30 bp, Artificial Sequence
  Primer
gtgaggatccatgtctatcagcaccctgcc

SEQ ID NO: 37 - DNA, 35 bp, Artificial Sequence
  Primer
gcgccctaggttacaaatcaacaatagcacatccc

SEQ ID NO: 38 - DNA, 29 bp, Artificial Sequence
  Primer
gagaggatccatgactcttgtggcatccg

SEQ ID NO: 39 - DNA, 33 bp, Artificial Sequence
  Primer
gcgccctaggttacaaatcatacaaccgcttgg

SEQ ID NO: 40 - DNA, 32 bp, Artificial Sequence
  Primer
gagaagatctatgacccaggctgctgctgaac

SEQ ID NO: 41 - DNA, 40 bp, Artificial Sequence
  Primer
gcgcactagtttacttgtcatgacggtcctttttgtaacc

SEQ ID NO: 42 - DNA, 36 bp, Artificial Sequence
  Primer
gagaggatccatgaccttttgcaacacgggagcaac

SEQ ID NO: 43 - DNA, 43 bp, Artificial Sequence
  Primer
gcgccctaggtcactcccgctccaaaacatagataatcttagc

SEQ ID NO: 44 - DNA, 30 bp, Artificial Sequence
  Primer
gcgcactagtatgtctatcagcaccctgcc

SEQ ID NO: 45 - DNA, 37 bp, Artificial Sequence
  Primer
gcgccccgggttacaaatcaacaatagcacatccctc

SEQ ID NO: 46 - DNA, 29 bp, Artificial Sequence
  Primer
gagaactagtatgactcttgtggcatccg

SEQ ID NO: 47 - DNA, 34 bp, Artificial Sequence
  Primer
gcgacccgggttacaaatcatacaaccgcttggc

SEQ ID NO: 48 - DNA, 29 bp, Artificial Sequence
  Primer
gagaactagtatgacccaggctgctgctg

SEQ ID NO: 49 - DNA, 35 bp, Artificial Sequence
  Primer
gagacccgggttacttgtcatgacggtcctttttg

SEQ ID NO: 50 - DNA, 36 bp, Artificial Sequence
  Primer
gcgcactagtatgaccttttgcaacacgggagcaac

SEQ ID NO: 51 - DNA, 42 bp, Artificial Sequence
  Primer
gcgcgaattctcactcccgctccaaaacatagataatcttag

SEQ ID NO: 52 - DNA, 20 bp, Artificial Sequence
  Primer
tcttcaactgctcctgtcct

SEQ ID NO: 53 - DNA, 21 bp, Artificial Sequence
  Primer
ctatcgtaatcgacaccatcc

SEQ ID NO: 54 - DNA, 20 bp, Artificial Sequence
  Primer
ccatcaaaaagctgaccaag

SEQ ID NO: 55 - DNA, 20 bp, Artificial Sequence
  Primer
atcgcagaagaggagggtag

SEQ ID NO: 56 - DNA, 19 bp, Artificial Sequence
  Primer
tggtgacgactacccctct

SEQ ID NO: 57 - DNA, 20 bp, Artificial Sequence
  Primer
cggttcctaggttccattct

SEQ ID NO: 58 - DNA, 20 bp, Artificial Sequence
  Primer
tccatctccctcaatggaac

SEQ ID NO: 59 - DNA, 20 bp, Artificial Sequence
  Primer
gccggtatagacctggaaac